(19) United States
(12) Patent Application Publication
(10) Pub. No.: US 2021/0052777 A1
(43) Pub. Date: Feb. 25, 2021

(54) METHODS AND COMPOSITIONS FOR TREATING AND PREVENTING DIABETES

(71) Applicants: TECHNION RESEARCH & DEVELOPMENT FOUNDATION LIMITED, Haifa (IL); B. G. NEGEV TECHNOLOGIES AND APPLICATIONS LTD., AT BEN-GURION UNIVERSITY, Beer Sheva (IL); RAMBAM MED-TECH LTD., Haifa (IL)

(72) Inventors: Shulamit LEVENBERG, Moreshet (IL); Eddy KARNIELI, Kiria-Tivon (IL); Eli Chaim LEWIS, Beer Sheva (IL); Margarita BECKERMAN, Migdal Haemek (IL); Chava HAREL, Haifa (IL)

(21) Appl. No.: 16/967,237

(22) PCT Filed: Feb. 4, 2019
(86) PCT No.: PCT/IL2019/050134

§ 371 Date: Aug. 4, 2020

Related U.S. Application Data
(60) Provisional application No. 62/626,098, filed on Feb. 4, 2018.

(51) Int. Cl. A61L 27/38; C12N 5/077; A61P 3/10

(52) U.S. Cl. CPC A61L 27/3834; A61P 3/10; C12N 5/0658

(57) ABSTRACT

The present invention is directed to, inter alia, a scaffold-cell construct including a biocompatible polymer (e.g., poly-l-lactic acid (PLLA) and polylactic glycolic acid (PLGA)) and recombinant cells having increased GLUT4 levels and/or activity. The invention is further directed to methods for reducing glucose levels in a subject in need thereof. Also provided are methods of producing the scaffold-cell construct of the invention.

### CROSS REFERENCE TO RELATED APPLICATIONS

This application claims the benefit of priority of U.S. Provisional Patent Application No. 62/626,098, filed Feb. 4, 2018, the contents of which are all incorporated herein by reference in their entirety.

### FIELD OF INVENTION

The present invention is in the field of biomedical engineering.

### BACKGROUND OF THE INVENTION

Diabetes mellitus (DM) is a disease that occurs in all populations and age groups and affects more than 10% of the Western world. It is the sixth leading cause of death in the United States, affecting between 6% and 7% of the US population equating to about 16 million people. The two most common general categories of this disease are termed type 1 diabetes (DM1) and type 2 diabetes (DM2). The number of people with diabetes is expected to reach 300 million in 2030, of which 90% will be DM2. Obesity is a major environmental factor contributing to the increasing incidence of DM2. Modern lifestyle, high-fat diet and lack of exercise were shown to trigger the development of DM2 in overweight patients with impaired glucose tolerance, while increased levels of markers and mediators of inflammation and oxidative stress components correlated with impaired insulin action (insulin resistance).

DM2 is a heterogeneous, polygenic disorder characterized by defects in insulin action in tissues (insulin resistance) and/or defects in pancreatic insulin secretion (beta cell dysfunction), which eventually results in loss of pancreatic insulin-secreting (beta) cells. The associated complications of diabetes are cardiovascular disease, peripheral vascular disease, stroke, diabetic neuropathy, diabetic nephropathy and diabetic retinopathy. These result in increasing disability, reduced life expectancy and enormous health costs. The development of these diabetes-related complications can be significantly reduced, partially prevented and retarded with control of blood glucose levels as close to normal as possible. However, in spite of the current knowledge and new treatment protocols, many patients currently do not reach the desired treatment goals. DM2 is a progressive and complex disorder that is difficult to treat effectively in the long term. The treatment begins with a well-balanced diet combined with exercise. Unfortunately, the majority of patients are unable to achieve or sustain near normo-glycemia without oral antidiabetic agents; a sizeable proportion of patients will eventually require oral and/or injectable hypoglycemic drugs and/or insulin therapy to maintain long-term glycemic control. The frequent need for escalating therapy reflects progressive loss of islet beta-cell function, usually in the presence of obesity-related insulin resistance.

Insulin resistance is a key component in the pathogenesis of DM2. It is a state of resistance to the action of insulin in its target tissues, i.e., impaired insulin stimulation of glucose transport in adipose tissue and skeletal muscle, and reduced inhibition of glucose production and release in the liver. One of the earliest defects detected in DM2 is reduction of cellular content and an impaired function of the insulin-responsive glucose transporter type 4, a member of the glucose transport proteins (GLUTs) mediating glucose uptake in eukaryotic cells. GLUT4 is the main glucose transporter regulating glucose entry from the blood into adipose and muscle tissues upon insulin stimulation.

### SUMMARY OF THE INVENTION

The present invention relates to transplantable scaffolds comprising cells comprising increased glucose transporter (e.g., GLUT4) levels and/or activity, such as for restoring glucose homoeostasis. The present invention further relates to scaffold-cell constructs for reducing elevated glucose levels, such as in subjects afflicted with diabetes and/or metabolic syndrome.

According to a first aspect, there is provided a scaffold-cell construct comprising: (a) porous scaffold comprising at least one biocompatible polymer; and (b) a first cell population deposited on or in said scaffold comprising recombinant cells having increased GLUT4 activity.

In some embodiments, the scaffold-cell construct comprises recombinant cells having increased GLUT4 content or activity sufficient for maintaining glucose homeostasis at levels of (i) less than or equal to 100 milligrams/Deciliter (mg/dL) at fasting; and (ii) less than 140 mg/dL postprandial. In some embodiments, the scaffold-cell construct comprises recombinant cells having increased GLUT4 activity sufficient for maintaining glucose homeostasis at levels of: (i) less than or equal to 120 milligrams/Deciliter (mg/dL) at fasting; and (ii) less than 160 mg/dL postprandial.

In some embodiments, the recombinant cells are induced to increase anyone of GLUT4 gene expression, membrane translocation (e.g., by targeting the cellular GLUT4 translocation machinery pathway) and insulin signal transduction. In some embodiments, insulin signal transduction inhibits GLUT4 degradation.

In some embodiments, the first cell population is selected from the group consisting of: skeletal myocyte-derived cell, cardiomyocyte-derived cell, adipocyte-derived cell, mesenchymal stem cell (MSC), embryonic stem cell (ESC), adult stem cell, differentiated ESC, differentiated adult Stem cell, and induced pluripotent Stem cell (iPSC).

In some embodiments, the first cell population is differentiated into myocyte, myoblast or myotube and any combination thereof. In some embodiments, the first cell population is selected from the group consisting of: differentiated myocytes, differentiated myoblasts differentiated myotubes and any combination thereof.

In some embodiments, there is provided a second cell population for maintaining growth and survival of the first cell population.

In some embodiments, the scaffold-cell construct comprises at least 1×10⁵ cells per mm³ of the at least one biocompatible polymer. In some embodiments, the biocompatible polymer comprises a polymer selected from a synthetic or natural material. In some embodiments, the biocompatible polymer comprises one or more polymers selected from the group consisting of poly-l-lactic acid (PLLA), polylactic glycolic acid (PLGA), and any combination or derivative thereof. In some embodiments, the biocompatible polymer comprises interconnected pores, wherein at least 80% of said pores have a diameter of between 200 and 600 microns. In some embodiments, PLLA and PLGA are in 3:1-1:3 w/w ratio.

In some embodiments, the scaffold-cell construct is for use in restoring glucose hemostatic levels in a diabetic subject in need thereof, wherein the restored glucose hemostatic levels are: (i) less than or equal to 100 mg/dL at fasting; and (ii) lower than 140 mg/dL postprandial. In some embodiments, the scaffold-cell construct is for use in restoring glucose hemostatic levels in a subject in need thereof, wherein the restored glucose hemostatic levels are: (i) less than or equal to 120 mg/dL at fasting; and (ii) lower than 160 mg/dL postprandial.

In some embodiments, the scaffold-cell construct is for use in treating or preventing a metabolic syndrome in a subject in need thereof. In some embodiments, the metabolic syndrome is selected from the group consisting of: obesity, diabetes mellitus, pre-diabetes and insulin resistance or related to insulin resistance.

In some embodiments, there is provided a composition comprising the scaffold-cell construct of the disclosed invention, and a pharmaceutically acceptable carrier.

According to another aspect, there is provided a scaffold-cell construct sufficient (or configured) to restore or maintain glucose levels of: (i) less than or equal to 100 mg/dL at fasting; and (ii) less than 140 mg/dL postprandial; the method comprising the steps of: (a) providing a scaffold comprising at least one biocompatible polymer; and (b) providing recombinant cells (e.g. solitary recombinant cells) having increased GLUT4 activity; thereby forming the scaffold-cell construct.

In some embodiments, the method further comprises a differentiation step (c) comprising seeding said recombinant cells on or in the scaffold for at least 7 days; thereby fully differentiating the recombinant cells into recombinant skeletal muscle cells having increased GLUT4 activity. In some embodiments, the method further comprises a validation step (d) comprising examining the ability of the scaffold-cell construct to maintain glucose homeostasis at levels of: (i) less than or equal to 100 mg/dL at fasting; and (ii) less than 140 mg/dL postprandial.

According to another aspect, there is provided a method for reducing glucose levels in a subject in need thereof, the method comprising the steps of: (a) providing a porous scaffold comprising at least one biocompatible polymer; and providing a first cell population deposited on or in the scaffold, wherein the first cell population comprises recombinant cells having increased GLUT4 activity, thereby providing a scaffold-cell construct; and (b) grafting the subject in need thereof with therapeutically effective amounts of the scaffold-cell construct, thereby reducing glucose levels in the subject.

In some embodiments, reducing glucose levels comprises reducing glucose levels to: (i) less than or equal to 120 mg/dL at fasting; and (ii) less than 160 mg/dL postprandial. In some embodiments, reducing glucose levels comprises reducing glucose levels to: (i) less than or equal to 100 mg/dL at fasting; and (ii) less than 140 mg/dL postprandial.

In some embodiments, grafting the subject is by autologous grafting or allogenous grafting. In some embodiments, the subject has glucose levels of: (i) greater than 100 mg/dL at fasting; and (ii) greater than or equal to 140 mg/dL postprandial.

In some embodiments, the method further comprises a step of validating restored glucose levels of: (i) less than or equal to 120 mg/dL at fasting; and (ii) less than 160 mg/dL postprandial; in the grafted subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a schematic presentation of a non-limiting example of the disclosed invention, by which, myoblasts are genetically-/molecularly-modified to over express GLUT4. The over-expressing GLUT4 myoblasts are cultured ex-vivo on 3D biocompatible scaffolds and transplanted thereafter.

FIGS. 2A-2F are representative immunofluorescent micrographs of whole poly-1-lactic acid (PLLA)/polylactic glycolic acid (PLGA) scaffolds seeded with 0.5×10⁶ L6 cells, either wild-type (L6WT; 2A, 2C and 2E) or GLUT4 over-expressing cells (L6GLUT4; 2B, 2D and 2F), and grown for 1 week in-vitro. Scaffolds were stained for different muscular markers, e.g., desmin (2A-2B), myosin heavy chain (MYH; 2C-2D), and myogenin (MYOG; 2E-2F) (all stained in red), all of which were shown to colocalize with the GLUT4 transporter (green; 2A-2F). Scale bar=500 μm.

FIGS. 3A-3F are representative immunofluorescent micrographs of whole poly-1-lactic acid (PLLA)/polylactic glycolic acid (PLGA) scaffolds seeded with 0.5×10⁶ L6 cells, either wild-type (L6WT; 3A, 3C and 3E) or GLUT4 over-expressing cells (L6GLUT4; 3B, 3D and 3F), and grown for 1 week in-vitro. Scaffolds were stained for different muscular markers, e.g., desmin (3A-3B), myosin heavy chain (MYH; 3C-3D), and myogenin (MYOG; 3E-3F) (all stained in red), all of which were shown to colocalize with the GLUT4 transporter (green; 3A-3F). Scale bar=100 μm.

FIGS. 4A-4H are representative immunofluorescent micrographs of whole poly-1-lactic acid (PLLA)/polylactic glycolic acid (PLGA) scaffolds seeded with 0.5×10⁶ L6 cells, either wild-type (L6WT; 4A, 4B, 4E and 4F) or GLUT4 over-expressing cells (L6GLUT4; 4C, 4D, 4G and 4H), and grown for 2 (4A, 4C, 4E and 4G) and 3 (4B, 4D, 4F and 4H) weeks in-vitro. Scaffolds were shown to comprise elongated, defined and aligned muscle fibers (stained in red using the muscular marker desmin). Round cell nuclei were also visualized (stained in blue using DAPI). Upper panel: scale bar=500 μm. Lower panel: scale bar=50 μm. (4E-4H) are representative enlargements of selected areas from (4A-4D), respectively.

FIG. 5 is vertical bar charts of 2-[3H]-deoxyglucose (2DOG) uptake rates in 0.25×10⁶ L6 cells, either wild-type (WT) or GLUT4 over-expressing cells (GLUT4) grown on poly-l-lactic acid (PLLA)/polylactic glycolic acid (PLGA) scaffolds for 3 weeks in-vitro (*p<0.05).

FIG. 6 is vertical bar charts of 2-[³H]-deoxyglucose (2DOG) uptake rates in 0.5×10⁶ L6 cells, either wild-type (WT) or GLUT4 over-expressing cells (GLUT4) grown on poly-l-lactic acid (PLLA)/polylactic glycolic acid (PLGA) scaffolds for 3 weeks in-vitro (*p<0.05; **p,0.01; ***p<0.005).

FIG. 7 is vertical bar charts of 2-[³H]-deoxyglucose (2DOG) uptake rates in 0.5×10⁶ L6 cells, either wild-type (WT) or GLUT4 over-expressing cells (GLUT4) grown on poly-l-lactic acid (PLLA)/polylactic glycolic acid (PLGA) scaffolds for 1, 2 or 3 weeks in-vitro (1 W, 2 W and 3 W, respectively). After each culturing time, cells were serum-starved, followed by incubation in the absence/presence of insulin (-basal/-ins, respectively). 2DOG uptake was then measured (*p<0.05; **p,0.01; ***p<0.005).

FIG. 8 is a graph demonstrating glycemic activity of immuno-deficient mice with no mature B and T cells (i.e., RAG), which bear a genetically defective insulin receptor (i.e., MKR) thus are insulin resistant and type 2 diabetes mellitus-afflicted, following glucose tolerance test (GTT). WT-FVBN mice, which are the background strain for RAG/MKR, were used as reference.

FIG. 9 is a graph of a glucose tolerance test (GTT), which was performed in RAG/MKR mice implanted with poly-l-lactic acid (PLLA)/polylactic glycolic acid (PLGA) scaffolds seeded with L6GLUT4 cells (OEG4 EMCs), mice implanted with empty scaffolds (Empty scfd), and non-implanted control mice (no scfd). Difference between no scfd and OEG4 * p<0.05; ** p<0.01, **** p<0.0001; Difference between Empty and OEG4 #p<0.05; ##p<0.01, ###p<0.001.

FIGS. 10A-10D are micrographs showing differentiation of C57-SC with different mediums, scale bar: 100 μm. (10A) BIO-AMF-2; (10B) DMEM 5% HS; (10C) DMEM 2% FBS; and (10D) SKMSM 2% FBS.

FIG. 11 is micrographs of enlarged images of myotubes formed out of differentiated C57-SC.

FIGS. 12A-12D are immunofluorescent micrographs of whole scaffold stain of PLLA\PLGA scaffolds seeded with 0.5×10⁶ (12A) or 1×10⁶ (12B) C57-SC cells grown 3 weeks in-vitro, stained for desmin (green), MYOG (magenta) and cell nuclei with DAPI (blue). (12C and 12D) are enlarged areas of (12A and 12B), respectively. Scale bar: 500 μm (12A and 12B); 50 μm (12C and 12D).

FIG. 13 is a vertical bar graph showing the quantification of desmin signal for whole scaffold stain of PLLA\PLGA scaffolds seeded with 0.5×10⁶ and 1×10⁶ C57-SC cells grown 3 weeks in-vitro.

FIGS. 14A-14B are immunofluorescent micrographs showing representative enlarged (20×) images of whole PLLA\PLGA scaffolds seeded with 0.5×10⁶ (14A) or 1×10⁶ (14B) C57-SC cells grown 3 weeks in-vitro, stained for desmin (green), MYOG (magenta) and cell nuclei with DAPI (blue), scale bar: 50 μm.

FIGS. 15A-15H are immunofluorescent micrographs showing staining for GLUT4 transporter (red) and cell nuclei with DAPI (blue) in C57-SC myotubes grown in 2D culture for 1 week, scale bar: 50 μm. Wild type (15A); Clones: PB A12 (15B), PB A34 (15C), PB A56 (15D), PB B12 (15E), RN A12 (15F), RN A34 (15G), RN A56 (15H).

FIG. 16 is a vertical bar graph showing GLUT4 signal intensity in C57-SC myotubes grown in 2D culture for 1 week. * p<0.05, **** p<0.0001.

FIGS. 17A-17H are immunofluorescent micrographs showing staining for GLUT4 transporter (red) and cell nuclei with DAPI (blue) in C57-SC myotubes grown in 2D culture for 1 week, scale bar: 50 μm. Wild type (17A); Clones: PB B34 (17B), PB B56 (17C), PB C12 (17D), RN B12 (17E), RN B34 (17F), RN B56 (17G), RN C12 (17H).

FIG. 18 is a vertical bar graph showing GLUT4 signal intensity in C57-SC myotubes grown in 2D culture for 1 week. *** p<0.001, **** p<0.0001.

FIGS. 19A-19H are immunofluorescent micrographs showing C57-SC myotubes grown in 2D culture for 1 week, stained for different myogenic markers and cell nuclei with DAPI. (19A-19D) are images of the following stains: desmin (green), MYOG (magenta) and cell nuclei (blue). (19E-19H) are images of the following stains: MYH (green), MYOG (magenta) and cell nuclei (blue). (19C and 19D) are enlarged areas of (19A and 19B), respectively. (19G and 19H) are enlarged areas of (19E and 19F), respectively. Scale bar: 250 μm (19A, 19B, 19E and 19F); 50 μm (19C, 19D, 19G and 19H). Clones: PB A12 (19A, 19C, 19E, and 19G); RN B12 (19B, 19D, 19F and 19H).

FIGS. 20A-20X are micrographs of 2D myotube formation of WT C57-SC. Scale bar: 100 μm. Cell number: (20A-20F) 50,000 cells; (20G-20L) 100,000 cells; (20M-20R) 175,000; and (20S-20X) 250,000. Days cultured: 1 day (20A, 20G, 20M and 20S); 3 days (20B, 20H, 20N and 20T); 4 days (20C, 20I, 20O and 20U); 5 days (20D, 20J, 20P and 20V); 7 days (20E, 20K, 20Q and 20W); and 10 days (20F, 20L, 20R and 20X).

FIGS. 21A-21X is micrographs of 2D myotube formation of RN-A56. Scale bar: 100 μm. Cell number: (21A-21F) 50,000 cells; (21G-21L) 100,000 cells; (21M-21R) 175,000; and (21S-21X) 250,000. Days cultured: 1 day (21A, 21G, 21M and 21S); 3 days (21B, 21H, 21N and 21T); 4 days (21C, 21I, 21O and 21U); 5 days (21D, 21J, 21P and 21V); 7 days (21E, 21K, 21Q and 21W); and 10 days (21F, 21L, 21R and 21X).

FIGS. 22A-22T is micrographs of 2D myotube formation of PB-A34. Scale bar: 100 μm. Cell number: (22A-22E) 50,000 cells; (22F-22J) 100,000 cells; (22K-22O) 175,000; and (22P-22T) 250,000. Days cultured: 1 day (22A, 22F, 22K and 22P); 2 days (22B, 22G, 22L and 22Q); 4 days (22C, 22H, 22M and 22R); 7 days (22D, 22I, 22N and 22S); and 10 days (22E, 22J, 22O and 22T).

FIG. 23 is a vertical bar graph showing 2-DOG uptake rates in C57-SC myotubes grown 1 week in 2D culture. * p<0.05, ** p<0.01, *** p<0.001, **** p<0.0001.

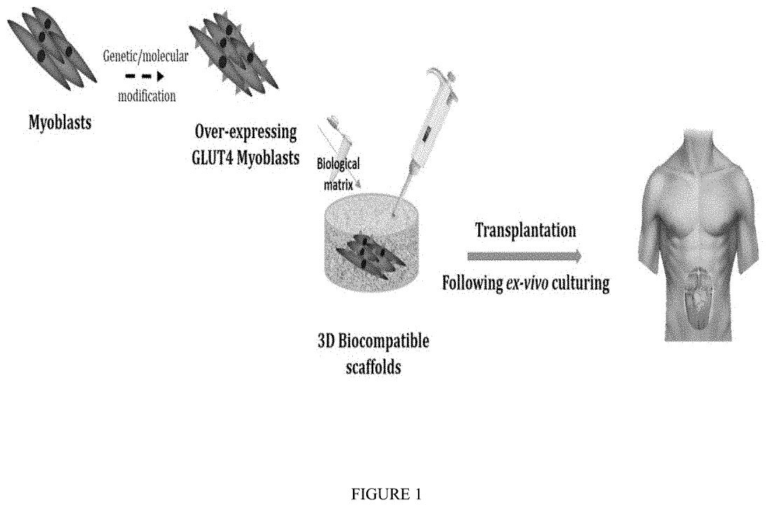

### DETAILED DESCRIPTION OF THE INVENTION

The present invention relates to transplantable scaffolds comprising cells having increased GLUT4 level and/or activity, such as for restoring glucose homoeostasis. The present invention further relates to scaffold-cell constructs for reducing hyperglycemia, such as in subjects afflicted with a metabolic syndrome.

The present invention is based, in part, on the finding that biocompatible scaffolds seeded with cells over-expressing GLUT4, restored normoglycemia. As demonstrated hereinbelow, scaffold-cell constructs specifically overexpressing GLUT4 were found to have greater glucose uptake rates compared with control cells in-vitro. As exemplified herein, glucose uptake rate was further enhanced when scaffold-cell constructs specifically over-expressing GLUT4 were incubated in the presence of insulin in-vitro. The invention is further based, in part, on the finding that scaffold-cell constructs specifically expressing GLUT4 restored normoglycemia when transplanted into insulin-resistant mice, thereby eliciting a therapeutic avenue relevant for treating diabetes and metabolic syndrome.

In some embodiments, the present invention provides a bioactive scaffold composition such that the bioactive scaffold composition supports systemic glucose homeostatic levels. In another embodiment, the present invention provides a bioactive scaffold such that the cells within maintain growth and survival at a site of transplantation.

As used herein, the terms “treat,” “treating,” “treatment,” and the like refer to reducing or ameliorating a disease or condition, e.g., diabetes, hyperglycemia, insulin resistance, and/or symptoms associated therewith. In some embodiments, treatment includes the partial or complete regeneration of normoglycemia in a subject. It will be appreciated that, although not precluded, treating a disease or condition does not require that the disease, condition, or symptoms associated therewith be completely eliminated.

As used herein, the term “normoglycemia” refers to the normal levels of glucose in the blood of healthy people. In some embodiments, normoglycemia is glucose levels of about 70 to 100 milligrams per deciliter (mg/dL) of blood in healthy people pre-meal (e.g., fasting). In some embodiments, normoglycemia is glucose levels of about 75 to 100 mg/dL of blood in healthy people pre-meal (e.g., fasting). In some embodiments, normoglycemia is glucose levels of about 85 to 100 mg/dL of blood in healthy people pre-meal (e.g., fasting). In some embodiments, normoglycemia is glucose levels of about 70 to 95 mg/dL of blood in healthy people pre-meal (e.g., fasting). In some embodiments, normoglycemia is glucose levels of about 75 to 90 mg/dL of blood in healthy people pre-meal (e.g., fasting).

In some embodiments, normoglycemia is glucose levels up to 140 mg/dL of blood in healthy people postprandial (e.g., 2 hours after eating). In some embodiments, normoglycemia is glucose levels of about 80 to 140 mg/dL of blood in healthy people postprandial (e.g., after eating). In some embodiments, normoglycemia is glucose levels of about 90 to 140 mg/dL of blood in healthy people postprandial (e.g., after eating). In some embodiments, normoglycemia is glucose levels of about 100 to 140 mg/dL of blood in healthy people postprandial (e.g., after eating). In some embodiments, normoglycemia is glucose levels of about 110 to 140 mg/dL of blood in healthy people postprandial (e.g., after eating). In some embodiments, normoglycemia is glucose levels of about 120 to 140 mg/dL of blood in healthy people postprandial (e.g., after eating). In some embodiments, normoglycemia is glucose levels of about 130 to 140 mg/dL of blood in healthy people postprandial (e.g., after eating). In some embodiments, normoglycemia is glucose levels less or equal to 140 mg/dL of blood in healthy people postprandial (e.g., 2 hours after eating).

The terms “glucose homeostatic levels”, “normoglycemia” and “normoglycemia” are interchangeable.

As used herein, the term “impaired fasting glucose (IFG)” refers to fasting glucose levels between 100-125 mg/dL.

In another embodiment, a glucose level lower than the mentioned herein normoglycemia is hypoglycemia. In another embodiment, a glucose level greater than the mentioned herein normoglycemia is considered hyperglycemia. In another embodiment, a subject having fasting blood glucose level of 100-125 mg/dL or 2 hours postprandial (e.g. test by glucose tolerance test) level of 140-199 mg/dL, is considered pre-diabetic and/or having insulin resistance.

In some embodiments, hyperglycemia of 200 mg/dL and above, without returning to basal levels within a period of 2 hours after a glucose tolerance test of 75 gr, is indicative of diabetes.

In some embodiments, glucose levels are measured by means of a blood test after fasting (e.g., FGT). In some embodiments, glucose levels are measured by means of an oral glucose tolerance test (e.g., OGTT). In some embodiments, glucose levels are estimated by the level of glycosylated hemoglobin (e.g., HbA_{1C}). As apparent to one skilled in the art, normoglycemia is having up to 5.7% HbA_{1C}. In another embodiment, hyperglycemia is having HbA_{1C} level of 6.5% or more. In another embodiment, HbA_{1C} level of 5.7-6.4% is indicative of prediabetes. In another embodiment, HbA_{1C} level of 6.5% or more is indicative of diabetes.

### Cells Over-Expressing Glucose Transporters

In some embodiments, the invention provides scaffold-cell constructs comprising a first cell population of recombinant cells having increased glucose transporter cellular content or activity.

In one embodiment, GLUT is GLUT 4. In some embodiments, the human GLUT 4 comprises a polynucleotide sequence according to accession number M20747.1. In some embodiments, the human GLUT 4 comprises a polypeptide sequence according to accession number AAA59189.1. In some embodiments, the murine GLUT 4 comprises a polynucleotide sequence according to accession number AB008453.1. In some embodiments, the human GLUT 4 comprises a polypeptide sequence according to accession number BAB03251.1. In one embodiment, GLUT is GLUT 1. In some embodiments, the human GLUT 1 comprises a polynucleotide sequence according to accession number NM_006516.3. In some embodiments, the human GLUT 1 comprises a polypeptide sequence according to accession number NP_006507.2.

As used herein, the term “a first cell population” refers to a skeletal myocyte-derived cell, a cardiomyocyte-derived cell, an adipocyte-derived cell, a mesenchymal stem cell (MSc), an embryonic stem cell (ESc), an adult stem cell, a differentiated ESc, a differentiated adult Stem cell, and an induced pluripotent Stem cell (iPSc). In another embodiment, a cell of the first cell population comprises any cell type capable of differentiating into a skeletal myocyte or a myotube.

In another embodiment, “skeletal muscle cell”, “skeletal myocyte”, “myoblast”, “myocyte” and “myotube” are used herein interchangeably.

Human embryonic pluripotent stem cells (hEPSCs) may be induced to differentiate into myogenic progenitor cells (iMPCs), by methods known in the art, such as described by Rao et al., (2018). Human induced pluripotent stem cells (iPS) may be induced to differentiate and form muscle fibers by methods known in the art, such as described by Chal et al., (2015). Human embryonic stem cells (hESCs) may be differentiated into skeletal myogenic cells as described by methods known in the art, such as by Shelton et al., (2014). Human mesenchymal stem cells (hMSCs) may be differentiated into skeletal myogenic cells by methods known in the art, such as described by Gang et al., (2004) or by Aboaloa and Han (2017). Human pluripotent stem cells (hPSCs) may be differentiated in vitro for generating muscle fibers and satellite-like cells methods known in the art, such as described by Chal et al., (2016).

In some embodiments, the first cell population comprises solitary cells. In some embodiments, the first cell population comprises at least 60%, 70%, 80%, 85%, 90% or 99% solitary cells, and any value and range therebetween. In some embodiments, the first cell population comprises 40-60%, 50-70%, 60-80%, 65-85%, 70-90% or 80-99% solitary cells. Each possibility represents a separate embodiment of the invention.

In some embodiments, the first cell population of the present invention expresses GLUT4 levels sufficient for maintaining glucose homeostasis at levels of less than or equal to 100 mg/dL at fasting. In another embodiment, the first cell population expresses GLUT4 levels sufficient for maintaining glucose homeostasis at levels of less than or equal to 110, 120 or 130 mg/dL at fasting. In another embodiment, fasting is for at least 1, 4, 8, 12 or 14 hours, and any range and value therebetween. In another embodiment, fasting is for 1-3 h, 2-5 h, 3-8 h, 4-6 h, 4-9 h, 7-12 h, 8-16 h, 14-20 h, 12-24 h. Each possibility represents a separate embodiment of the invention.

In some embodiments, the first cell population of the present invention expresses GLUT4 levels sufficient for maintaining glucose homeostasis at levels of less than 140 mg/dL postprandial. In another embodiment, the first cell population expresses GLUT4 levels sufficient for maintaining glucose homeostasis at levels of less than 150 or 160 mg/dL postprandial. In another embodiment, postprandial is not more than 15, 30, 45 or 60 min postprandial, and any value and range therebetween. In another embodiment, postprandial is not more than 2, 3, 4, 5 or 6 hours postprandial, and any value and range therebetween. In another embodiment, postprandial is 0.5-1.5 h, 1-3 h, 2-4 h, 3-5 h, or 3-7 h postprandial. Each possibility represents a separate embodiment of the invention.

In some embodiments, the first cell population of the present invention has increased glucose responding activity or glucose sensitivity compared to control cells. In some embodiments, the first cell population has increased glucose responding activity or glucose sensitivity compared to control cells in the absence of insulin (e.g., basal). In some embodiments, the first cell population has increased glucose responding activity or glucose sensitivity compared to control cells in the presence of insulin. In some embodiments, the first cell population has increased glucose responding activity or glucose sensitivity compared to control cells in the absence or presence of insulin. In some embodiments, the increased glucose responding activity or glucose sensitivity of the first cell population results in increased rates of glucose uptake, increased amounts of glucose taken by the cells, increased rates of glucose metabolism, increased amounts of metabolized glucose, and any combination thereof, all compared to control cells under basal conditions (i.e., in the absence of insulin) or in the presence of insulin, or any combination thereof.

As would be apparent to one skilled in the art, fully differentiated skeletal muscle cells are characterized by positive immune-stain for myoblasts' differentiation and structural markers. Non-limiting examples of myoblasts' differentiation markers include MYOG (differentiation marker), and desmin and MyH (structural markers). As well known to a skilled artisan, fully differentiated skeletal muscle cells are observed by microscopy as organized multinucleated myotubes.

In some embodiments, the scaffold-cell constructs further comprise a second cell population, such as for supporting the viability of the first cell population.

As used herein, the term “a second cell population” refers to a cellular substrate that may be included in the disclosed scaffolds. In another embodiment, the cellular substrate is any cellular substrate. In some embodiments, the cellular substrate promotes growth of the first cell population. In some embodiments, the cellular substrate promotes the survival of the first cell population. In some embodiments, the cellular substrate secrets growth factors. In some embodiments, the cellular substrate secrets cytokines. In some embodiments, the cellular substrate secrets fibroblast growth factors (FGFs). In some embodiments, the cellular substrate produces extracellular matrix proteins. In some embodiments, the cellular substrate produces integrins. None limiting examples of the cellular substrate include an endothelial cell, a fibroblast cell, a Schwann cell, an oligodendrocyte, an olfactory ensheathing glia (OEG), an oligodendrocyte progenitor cell (OPC), a macrophage, or any combination thereof.

In some embodiments, the first cell population of the invention secretes biomolecules which promote its own growth, differentiation, survival, or any combination thereof. In some embodiments, the second cell population of the present invention secretes biomolecules which promote the first cell population's growth, differentiation, survival, or any combination thereof. As used herein, biomolecules include, but not limited to, myokines, hormones, cytokines, cyclin-dependent kinases, cell cycle regulatory proteins, chaperonins, and others.

As used herein, the term “myokine” refers to any peptide or polypeptide, bioactive lipid, second messenger, etc. derived from differentiated muscle cells. In one embodiment, fully differentiated skeletal muscle cells derived from the first cell population of the invention secrete a myokine.

As used herein, the term “hormone” refers to a member of a group of signaling molecules which promote (e.g., stimulate and/or inhibit) a physiological reaction in a target site. In some embodiments, the hormone is an endocrine hormone, i.e., transported through the circulatory system to distant target sites. In some embodiments, the hormone is a paracrine hormone, i.e., has a target site in its vicinity, which may not require transport through the circulatory system but may be via diffusion. In some embodiments, the hormone is an autocrine hormone, i.e., has itself as a target for signaling. In some embodiments, a hormone is proteinaceous hormone (e.g., comprised of amino acids and or derivatives such as, amines, protein derivatives, peptides, polypeptides and proteins). In some embodiments, a hormone is an eicosanoid hormone (e.g., metabolized from a polyunsaturated fatty acid). In some embodiments, a hormone is a steroid hormone.

As used herein, the term “cytokine” refers to cell signaling immunomodulating molecule comprising a small peptide of approximately 20 kDa or less. Non-limiting examples of a cytokine include chemokine, interferon, interleukin, lymphokine, and tumor necrosis factor.

As used herein, the term “cyclin-dependent kinase” refers to a member of the family of kinases (i.e., enzymes capable of phosphorylating other polypeptides) of approximately 30 to 40 kDa involved in the regulation of the cell cycle. In some embodiments, the cyclin dependent kinase (CDK) is selected from CDCl₂ (CDK1), CDK2, CDK4, CDK5, CDK6, CDK7, CDK8, or others.

As used herein, the term “cell cycle regulatory protein” refers to a polypeptide of the “cell cycle regulatory (CCR)-protein family, which functions in one of either role of an agonist of cell-cycle regulation or an antagonist of cell-cycle regulation. In some embodiments, a CCR-protein has specific binding affinity to a CDK. In some embodiments, the CCR-protein inhibits proliferation/cell growth of a cell. A CCR protein-family member is selected from E2F, p13.5, p15, p16, p21, p²⁷, p53, or others.

As used herein, the term “chaperonin” refers to a member of a sub-group of chaperones, which are proteins that utilize energy to provide proper conditions for correct folding of other proteins (e.g., preventing aggregation). A chaperonin may include the GroEL/GroES complex, TRiC/CCT and Mm cpn.

Methods for determining the presence or level of any one of myokines, hormones, cytokines, cyclin dependent kinases, cell cycle regulatory proteins, or chaperonins as disclosed hereinabove in a biological sample are common and would be apparent to one of ordinary skill in the art. Non-limiting examples include, but are not limited to, antibody arrays, spectroscopy, column chromatography; HPLC; FPLC; matrix-affinity chromatography; reverse-phase chromatography; optical spectroscopic techniques; electrophoretic separation; or others.

Methods of evaluating cell growth and survival would be apparent to the skilled artisan, non-limiting examples of which include ATP test, Calcein AM, Clonogenic assay, Ethidium homodimer assay, Fluorescein diacetate hydrolysis/Propidium iodide staining (FDA/PI staining), Flow cytometry, Formazan-based assays (MTT/XTT), Trypan Blue, TUNEL assay, or others.

As described herein, a first cell population may be seeded on a scaffold of the invention, in vitro. In another embodiment, a first cell population which is seeded on scaffolds of the invention exhibits insulin-responsive properties. In another embodiment, the insulin responsive properties are enhanced glucose uptake. In another embodiment, glucose uptake is enhanced by at least by 2-fold, 3-fold, 4-fold, 5-fold, 6-fold, 8-fold or 10-fold, and any value and range therebetween. In some embodiments, glucose uptake is enhanced by 50-100%, 100-250%, 200-500%, 300-700%, 650-900%, or 750-1,500%. Each possibility represents a separate embodiment of the invention.

In some embodiments, cellular glucose uptake is attributed to the activity of GLUT4.

As used herein, “increased GLUT4 activity” includes, but not is limited to, increase of anyone of: GLUT4 gene expression, GLUT4 cellular content, membrane translocation by the cellular translocation machinery pathway, insulin signal transduction, glucose sensitivity in the absence or presence of insulin, or any combination thereof. In some embodiments, increased gene expression includes, but is not limited to, increased amount of the gene's mRNA molecules, increased amount of the translated polypeptides, or any combination thereof. In some embodiments, increased GLUT4 activity enhances cellular insulin sensitivity. In some embodiments, increased GLUT4 activity enhances cellular glucose uptake. In some embodiments, increased GLUT4 activity enhances cellular insulin sensitivity and cellular glucose uptake.

In some embodiments, insulin signal transduction inhibits GLUT4 degradation. In one embodiment, GLUT4 is degraded by a proteasome dependent pathway. In another embodiment, GLUT4 is degraded by an oxidative stress mediated pathway. Degradation of GLUT4 can be determined by any method known in the art, including, but not limited to, methods utilizing specific anti GLUT4 antibodies, comprising anti ubiquitinated-GLUT4 antibodies, among others. Non-limiting examples of methods which utilize antibodies include, but are not limited to, sandwich enzyme linked immunosorbent assay (ELISA, e.g., of either tissue homogenates, cell lysate or other biological fluids), 26S proteasome degradation assay, immunoprecipitation, immune-blotting, immune-histochemistry, immune-cytochemistry, any combination thereof, or any other method known to one of ordinary skill in the art.

In some embodiments, GLUT4 activity is increased in the first cell population by at least 2-fold, 5-fold, 10-fold, 25-fold or 100-fold, and any value and range therebetween. In another embodiment, GLUT4 activity is increased in the first cell population by 5-50%, 20-100%, 75-250%, 200-500%, 450-750%, or 600-1,000%. Each possibility represents a separate embodiment of the invention.

As used herein, the first cell population of the invention comprises a recombinant cell. In some embodiments, the term “recombinant cell” used herein, refers to a cell whose genetic composition was modified. In one embodiment, a recombinant cell comprises exogenous polynucleotide. In another embodiment, a recombinant cell expresses an exogenous polynucleotide. In one embodiment, a recombinant cell constitutively expresses an endogenous polynucleotide. In another embodiment, a recombinant cell conditionally expresses an endogenous polynucleotide. A non-limiting example of constitutive expression is achieved by contacting a cell with an endogenous polynucleotide operably linked to a constantly operating promoter polynucleotide. In another embodiment, recombinant cell facultatively expresses endogenous or exogenous polynucleotide in response to a specific stimulation (e.g., induced or conditional expression). In another embodiment, recombinant cell expresses endogenous or exogenous polynucleotide indefinitely. Recombinant expressions systems are well known to one skilled in the art, non-limiting examples of which include the Tetracycline-controlled transcriptional activation (“Tet-on/Tet off”), Actin-GAL4-UAS, IPTG-inducible conditional expression, or others.

In another embodiment, the expression of the GLUT4 gene in the recombinant first cell population grown on a scaffold of the invention is upregulated by at least 2-fold, 3-fold, 4-fold, 5-fold, 6-fold, 8-fold or 10-fold, and any value and range therebetween. In another embodiment, the expression of the GLUT4 gene in the recombinant first cell population grown on a scaffold of the invention is upregulated by 5-50%, 40-100%, 75-250%, 200-350%, 300-500%, 400-750%, or 700-1,500%. Each possibility represents a separate embodiment of the invention.

In another embodiment, increased GLUT4 activity is in the level sufficient to restore glucose homeostasis. In another embodiment, increased GLUT4 activity is in the level sufficient to maintain glucose homeostasis. In another embodiment, increased GLUT4 activity is in the level sufficient to rectify glucose homeostasis. In another embodiment, increased GLUT4 activity is in the level sufficient to reduce hyperglycemia in a subject. In another embodiment, increased GLUT4 activity is in the level sufficient to restore normoglycemia in a subject afflicted with hyperglycemia. In another embodiment, increased GLUT4 activity is in the level sufficient to reduce fasting glucose levels to levels less than or equal to 80 mg/dL in a subject. In another embodiment, increased GLUT4 activity is in the level sufficient to reduce fasting glucose levels to levels less than or equal to 90 mg/dL in a subject. In another embodiment, increased GLUT4 activity is in the level sufficient to reduce fasting glucose levels to levels less than or equal to 95 mg/dL in a subject. In another embodiment, increased GLUT4 activity is in the level sufficient to reduce fasting glucose levels to levels less than or equal to 100 mg/dL in a subject. In another embodiment, increased GLUT4 activity is in the level sufficient to reduce fasting glucose levels to levels less than or equal to 105 mg/dL in a subject. In another embodiment, increased GLUT4 activity is in the level sufficient to reduce fasting glucose levels to levels less than or equal to 110 mg/dL in a subject. In another embodiment, increased GLUT4 activity is in the level sufficient to reduce fasting glucose levels to levels less than or equal to 115 mg/dL in a subject. In another embodiment, increased GLUT4 activity is in the level sufficient to reduce fasting glucose levels to levels less than or equal to 120 mg/dL in a subject. In another embodiment, increased GLUT4 activity is in the level sufficient to reduce postprandial glucose levels to levels less than 135 mg/dL in a subject. In another embodiment, increased GLUT4 activity is in the level sufficient to reduce postprandial glucose levels to levels less than 140 mg/dL in a subject. In another embodiment, increased GLUT4 activity is in the level sufficient to reduce postprandial glucose levels to levels less than 145 mg/dL in a subject. In another embodiment, increased GLUT4 activity is in the level sufficient to reduce postprandial glucose levels to levels less than 150 mg/dL in a subject. In another embodiment, increased GLUT4 activity is in the level sufficient to reduce postprandial glucose levels to levels less than 155 mg/dL in a subject. In another embodiment, increased GLUT4 activity is in the level sufficient to reduce postprandial glucose levels to levels less than 160 mg/dL in a subject.

In some embodiments, a recombinant cell of the present invention comprises a cell in which GLUT4 levels have been directly elevated. As used herein, the term “directly elevated levels of GLUT4” refers to contacting a cell with a polynucleotide comprising a GLUT4 encoding sequence and inducing its expression, thereby resulting in its elevated levels in the cell. In some embodiments, the elevated levels are increased levels of the GLUT4 encoding gene transcription. In some embodiments, the elevated levels are increased amounts of the GLUT4 mRNA molecules. In some embodiments, the elevated levels are increased rates of the GLUT4 mRNA translation. In some embodiments, the elevated levels are increased GLUT4 mRNA stability. In some embodiments, the elevated levels are increased amounts of the GLUT4 polypeptide. In some embodiments, the elevated levels are achieved by a vector or a plasmid transfection. In some embodiments, the vector or plasmid is transfected to a cell of the invention. In some embodiments, the vector comprises a polynucleotide comprising GLUT4 encoding sequence. In some embodiments, the increased levels of the GLUT4 encoding gene are induced by GLUT4 gene editing. In some embodiments, the gene editing comprises molecular alterations in the GLUT4 genomic polynucleotide's sequence which induce or promotes the gene's over expression. In some embodiments, the gene editing is achieved by Clustered Regularly Interspaced Short Palindromic Repeats (CRISPR) system.

In some embodiments, a recombinant cell of the invention comprises a cell in which GLUT4 levels have been indirectly elevated. As used herein, the term “indirectly elevated levels of GLUT4” refers to blocking negative regulators inhibiting GLUT4 activity, thereby resulting in its elevated activity. In some embodiments, a negative regulator is a transcription inhibitor. In some embodiments, a negative regulator is a translation inhibitor. In some embodiments, a negative regulator inhibits GLUT4 migration via the secretory pathway. In some embodiments, a negative regulator inhibits trafficking of the GLUT4 polypeptide to the cellular membrane. In some embodiments, a negative regulator is an antibody. In some embodiments, a negative regulator is an RNA molecule. In some embodiments, a negative regulator is an antisense RNA molecule. In some embodiments, a negative regulator is a micro RNA molecule (miRNA). In some embodiments, a negative regulator is a protease inhibitor. In some embodiments, a negative regulator is steroid.

The term “polynucleotide” refers to a nucleic acid (e.g., DNA or RNA) sequence that comprises coding sequences necessary for the production of a polypeptide. In one embodiment, a polynucleotide refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA dependent DNA polymerase. In one embodiment, the sequence can be subsequently amplified in vivo or in vitro using a DNA polymerase.

In one embodiment, “genomic polynucleotide sequence” refers to a sequence derived (isolated) from a chromosome and thus it represents a contiguous portion of a chromosome.

In some embodiments, the molecule blocking a negative regulator inhibiting GLUT4 activity is a monoclonal antibody. In some embodiments, the molecule blocking a negative regulator inhibiting GLUT4 activity is a recombinant monoclonal antibody. In some embodiments, the molecule blocking a negative regulator inhibiting GLUT4 activity is a polyclonal antibody. In some embodiments, the molecule blocking a negative regulator inhibiting GLUT4 activity is a recombinant polyclonal antibody.

According to the method disclosed herein, in some embodiments thereof, the molecule blocking a negative regulator inhibiting GLUT4 activity is a nucleic acid. In some embodiments, the molecule blocking a negative regulator inhibiting GLUT4 activity has one or more chemical modifications to the backbone or side chains as described herein. In some embodiments, the molecule blocking a negative regulator inhibiting GLUT4 activity is a RNA interfering (RNAi) molecule. In some embodiments, the interfering RNA is a small hairpin RNA (shRNA), a small interfering RNA (siRNA), a double stranded RNA (dsRNA), or a miRNA antagonizing RNA (antagomiR). According to the method disclosed herein, in some embodiments thereof, blocking a negative regulator inhibiting GLUT4 expression and/or activity is by means of the CRISPR Cas system.

Inhibitory nucleic acids useful in the present methods and compositions, in some embodiments thereof, include antisense oligonucleotides, ribozymes, external guide sequence (EGS) oligonucleotides, siRNA compounds, single- or double-stranded RNAi compounds such as siRNA compounds, modified bases/locked nucleic acids (LNAs), antagomirs, peptide nucleic acids (PNAs), or other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, siRNA; a micro RNA (miRNA); a small temporal RNA (stRNA); shRNA; small RNA-induced gene activation (RNAa); small activating RNAs (saRNAs), or combinations thereof.

As used herein, the term “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

As would be apparent to one of ordinary skill in the art, a CRISPR Cas system as can be used according to the disclosed method, utilizes a CRISPR complex binding to a polynucleotide target, such that the binding results in increased or decreased expression of the polynucleotide. In some embodiments, the method further comprises delivering one or more vectors to the cells of the invention, wherein the one or more vectors drive expression of one or more of: the CRISPR enzyme, the guide sequence linked to the tracer mate sequence, or the tracer sequence.

The inhibitory nucleic acids useful according to the herein disclosed method have at least 80% sequence complementarity to a target region within the target nucleic acid, e.g., 90%, 95%, or 100% sequence complementarity to the target region within the targeted gene, and any value and range therebetween. Each possibility represents a separate embodiment of the invention.

In some embodiments, the molecule blocking a negative regulator inhibiting GLUT4 activity is a peptide mimetic or peptidomimetic. The terms “peptide mimetics” or “peptidomimetics” as used herein, refer to structures which serve as substitutes for peptides in interactions between molecules (Morgan et al., 1989). Peptide mimetics include synthetic structures which may or may not contain amino acids and/or peptide bonds but retain the structural and functional features of a peptide, or agonist or antagonist (i.e. enhancer or inhibitor) of the invention. Peptide mimetics also include peptoids, oligopeptoids (Simon et al., 1972); and peptide libraries containing peptides of a designed length representing all possible sequences of amino acids corresponding to a motif, peptide, or agonist or antagonist (i.e. enhancer or inhibitor) of the invention.

In one embodiment, the present invention provides a vector or a plasmid comprising the nucleic acid molecule as described herein. In one embodiment, a vector or a plasmid is a composite vector or plasmid. In one embodiment, a vector or a plasmid is a man-made vector or plasmid comprising at least one DNA sequence which is artificial. In one embodiment, the present invention provides a vector or a plasmid comparing: pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In one embodiment, the present invention provides a vector or a plasmid comprising regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Typically, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

In another embodiment, the first cell population occupies the scaffold in all three dimensions. In another embodiment, any cell mentioned herein occupies the scaffold in all three dimensions. In another embodiment, the first cell population occupies the pores. In another embodiment, the first cell population resides within a pore. In another embodiment, the first cell population resides on the scaffold's surface. In another embodiment, the first cell population is present both within the pores and on the scaffold's surface. In some embodiments, cells as mentioned herein occupies at least 25%, at least 35%, at least 45%, at least 60%, at least 75%, at least 85%, at least 90%, at least 95%, or at least 99% of areas of the scaffold as disclosed herein, for example, pores, surface, or both, and any value and range therebetween. In some embodiments, cells as mentioned herein occupies 20-30%, 25-35%, 25-45%, 40-60%, 50-75%, 70-85%, 75-90%, 80-95%, 92-100% of areas of the scaffold as disclosed herein, for example, pores, surface, or both. Each possibility represents a separate embodiment of the invention.

In another embodiment, the cells are autologous cells. In another embodiment, the cells are allogeneic cells.

In another embodiment, the first cell population expresses one or more markers selected from desmin, myosin heavy chain (MYH), or myogenin (MYOG). None limiting examples for methods for detecting such markers are disclosed hereinbelow and would be apparent to a skilled artisan.

### Scaffolds

As used herein, the term “scaffold” refers to a structure comprising a biocompatible material that provides a surface suitable for adherence, attachment, anchoring, maturation, differentiation, proliferation, or any combination thereof, of cells. A scaffold may further provide mechanical stability and support. A scaffold may be in a particular shape or form so as to influence or delimit a three-dimensional shape or form assumed by a population of proliferating cells. As used herein three-dimensional shapes include: films, ribbons, cords, sheets, flat discs, cylinders, spheres, 3-dimensional amorphous shapes, or others.

As used herein, the term “biocompatible”, in some embodiments, refers to the ability of an object to be accepted by and to function in a recipient without eliciting a significant foreign body response (such as, for a non-limiting example, an immune, inflammatory, thrombogenic, or the like response). For example, when used with reference to one or more of the polymeric materials of the invention, biocompatible refers to the ability of the polymeric material (or polymeric materials) to be accepted by and to function in its intended manner in a recipient.

The scaffold, in one embodiment, is a porous matrix. In another embodiment, the porous scaffold comprises at least 50% porosity. In another embodiment, the porous scaffold comprises at least 60% porosity, at least 70% porosity, at least 75% porosity, at least 80% porosity, at least 85% porosity, at least 90% porosity, at least 92% porosity, or at least 95% porosity, and any value and range therebetween. In another embodiment, the porous scaffold comprises 45-55% porosity, 50-70% porosity, 60-80% porosity, 75-90% porosity, or 80-97% porosity. Each possibility represents a separate embodiment of the invention.

In another embodiment, the porous scaffold comprises pores having a diameter of at least 100 μm. In another embodiment, the porous scaffold comprises pores having a diameter of at least 120 μm. In another embodiment, the porous scaffold comprises pores having a diameter of at least 150 μm. In another embodiment, the porous scaffold comprises pores having a diameter of 100-900 μm. In another embodiment, the porous scaffold comprises pores having a diameter of 120-900 μm. In another embodiment, the porous scaffold comprises pores having a diameter of 120-850 μm. In another embodiment, the porous scaffold comprises pores having a diameter of 150-800 μm. In another embodiment, the porous scaffold comprises pores having a diameter of 200-800 μm. In another embodiment, the porous scaffold comprises pores having a diameter of 220-750 μm.

In another embodiment, the scaffold (e.g., matrix) is devoid of any one of an organized structure, layer, or network of layers. In another embodiment, the composition is devoid of any layer of aligned fibers. In another embodiment, the scaffold is devoid of any layer of aligned fibers. In another embodiment, the composition is devoid of curved fibers. In another embodiment, the scaffold is devoid of curved fibers.

In another embodiment, the composition described herein comprises poly-1-lactic acid (PLLA). In another embodiment, the composition described herein comprises polylactic glycolic acid (PLGA). In another embodiment, the composition described herein comprises both poly-l-lactic acid (PLLA) and polylactic glycolic acid (PLGA). In another embodiment, the scaffold described herein comprises both poly-l-lactic acid (PLLA) and polylactic glycolic acid (PLGA). In another embodiment, PLLA and PLGA are in 1:3 to 3:1 w/w ratio. In another embodiment, PLLA and PLGA are in 1:2 to 2:1 w/w ratio. In another embodiment, PLLA and PLGA are in 1:1.5 to 1.5:1 w/w ratio. In another embodiment, PLLA and PLGA are in 1:1 w/w ratio.

In another embodiment, a composition as described herein is cultured for at least 14 days in-vitro or ex-vivo, in order to reach baseline proliferation rates.

In another embodiment, the cell is attached to a scaffold such as described herein for at least 7 days. In another embodiment, the cell is attached to a scaffold such as described herein for at least 14 days. In another embodiment, the cell is attached to a scaffold such as described herein for 7 to 21 days. In another embodiment, the cell is attached to a scaffold such as described herein for 14 to 31 days. In another embodiment, the cell is attached to a scaffold such as described herein for 30 to 60 days. In another embodiment, the cell is attached to a scaffold such as described herein for 25 to 75 days. In another embodiment, the cell is attached to a scaffold such as described herein for 50 to 90 days.

In another embodiment, the present invention is further directed to a composition that is cultured for at least 7 days. In another embodiment, the composition is cultured for at least 14 days. In another embodiment, the composition is cultured for at least 21 days. In another embodiment, the composition is cultured for at least 28 days. In another embodiment, the composition is cultured for at least 3 months days. In another embodiment, the composition is cultured for 5-8 days, 7-15 days, 14-28 days, 21-35 days, 1-2 months, 2-3 months, or 4-6 months. Each possibility represents a separate embodiment of the invention.

In another embodiment, the porous scaffold of the invention is further coated with a polymer. In another embodiment, the porous scaffold is further coated with an extracellular matrix protein. In another embodiment, the porous scaffold is further coated with fibronectin. In another embodiment, the porous scaffold is further coated with polypyrrole. In another embodiment, the porous scaffold is further coated with polycaprolactone. In another embodiment, the porous scaffold is further coated with poly(ethersulfone). In another embodiment, the porous scaffold is further coated with poly(acrylonitrile-co-methylacrylate) (PAN-MA). In another embodiment, the porous scaffold further comprises a chemoattractant, such as, but not limited to laminin-1.

In another embodiment, a composition as described herein further comprises fibrin. In another embodiment, a composition as described herein further comprises thrombin.

In another embodiment, a scaffold such as described herein is 10-160 mm3. In another embodiment, a scaffold such as described herein is 10-80 mm3. In another embodiment, a scaffold such as described herein is 15-50 mm3.

In another embodiment, the scaffolds described herein can further include a therapeutic agent (e.g., suitable for treating a subject afflicted with a metabolic disease). In another embodiment, the therapeutic agent comprises any therapeutic agent. In another embodiment, the therapeutic agent comprises a polypeptide, polypeptide fragment, nucleic acid molecule, small molecule, ribozyme, shRNA, RNAi, antibody, antibody fragment, scFv, enzyme, carbohydrate, or any combination thereof. In some embodiments, the therapeutic agent comprises glucose transporter type 4 (GLUT4), also known as either facilitated glucose transporter member 4 or solute carrier family 2. The scaffold described herein can release, in one embodiment, the therapeutic agent for at least 1 day, at least 1 week, or at least 1 month, and any value and range therebetween. The scaffold described herein can release, in another embodiment, the therapeutic agent for 1 to 5 days, 4 to 8 days, 1 to 3 weeks, 2 to 5 weeks, 1 to 3 months. Each possibility represents a separate embodiment of the invention.

In another embodiment, a composition as described herein further comprises a material selected from collagen-GAG, collagen, fibrin, PLA, PGA, PLA-PGA co-polymer, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, nonbiodegradable polyurethane, polyurea, poly(ethylene vinyl acetate), polypropylene, polymethacrylate, polyethylene, polycarbonate, poly(ethylene oxide), or any combination thereof.

In another embodiment, a composition as described herein further comprises a cell adhesion promoting agent, a proliferation inducer, a differentiation inducer, an extravasation inducer and/or a migration inducer. In another embodiment, a composition as described herein further comprises a cell adhesion protein, a growth factor, a cytokine, a hormone, a protease a protease substrate, or any combination thereof. In another embodiment, any substance as described herein is attached to the scaffold. In another embodiment, any substance as described herein is embedded within the scaffold. In another embodiment, any substance as described herein is impregnated within the scaffold. In another embodiment, a scaffold such as described herein is coated with a gel. In another embodiment, a scaffold such as described herein is biodegradable.

In another embodiment, the porosity of the scaffold is controlled by a variety of techniques known to those skilled in the art. In another embodiment, as the porosity is increased, use of polymers having a higher modulus, addition of suffer polymers as a co-polymer or mixture, or an increase in the cross-link density of the polymer are used to increase the stability of the scaffold with respect to cellular contraction.

In another embodiment, the choice of polymer and the ratio of polymers in a co-polymer scaffold of the invention is adjusted to optimize the stiffness/porosity of the scaffold. In another embodiment, the molecular weight and cross-link density of the scaffold is regulated to control both the mechanical properties of the scaffold and the degradation rate (for degradable scaffolds). In another embodiment, the mechanical properties are optimized to mimic those of the tissue at the implant site. In another embodiment, the shape and size of the final scaffold are adapted for the implant site and tissue type. In another embodiment, scaffold materials comprise natural or synthetic organic polymers that can be gelled, or polymerized or solidified (e.g., by aggregation, coagulation, hydrophobic interactions, or cross-linking) into a hydrogel e.g., structure that entraps water and/or other molecules.

In another embodiment, polymers used in scaffold material compositions are biocompatible, biodegradable and/or bioerodible and act as adhesive substrates for cells. The term “biodegradable polymer” as used herein, refers to a polymer or polymers which degrade in-vivo, and wherein erosion of the polymer or polymers over time occurs concurrent with or subsequent to release of cells/tissue. The terms “biodegradable” and “bioerodible” are equivalent and are used interchangeably herein.

In another embodiment, the structural scaffold materials are non-resorbing or non-biodegradable polymers or materials. The term “non-biodegradable polymer”, as used herein, refers to a polymer or polymers which at least substantially (i.e., 50% or more) do not degrade or erode in-vivo. The terms “non-biodegradable” and “non-resorbing” are equivalent and are used interchangeably herein.

In another embodiment, scaffold materials comprise naturally occurring substances, such as, fibrinogen, fibrin, thrombin, chitosan, collagen, alginate, poly(N-isopropylacrylamide), hyaluronate, albumin, collagen, synthetic polyamino acids, prolamines, polysaccharides such as alginate, heparin, and other naturally occurring biodegradable polymers of sugar units. In another embodiment, structural scaffold materials are ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations.

In another embodiment, the scaffolds of the invention are made by any of a variety of techniques known to those skilled in the art. Salt-leaching, porogens, solid-liquid phase separation (sometimes termed freeze-drying), and phase inversion fabrication are used, in some embodiments, to produce porous scaffolds.

As used herein, “transplanting” refers to providing the scaffold supported cells of the present invention (e.g., scaffold-cell construct), using any suitable route, as known to one skilled in the art. In one embodiment, the scaffold supported cells are administered by injection using a catheter.

In another embodiment, skeletal myocytes- or cardiomyocytes- or adipocytes-derived cells on scaffolds induce insulin sensitivity behavior, including maintaining proper glucose homeostasis. In another embodiment, the composition comprising PLLA/PLGA scaffolds seeded with GLUT4 overexpressing skeletal myocytes- or cardiomyocytes- or adipocytes-derived cells dramatically and unexpectedly increased the therapeutic potential of transplantation in patients afflicted with a metabolic syndrome, including but not limited to obesity, pre-diabetes, Diabetes mellitus type 2, insulin resistance or related to insulin resistance, among others.

In another embodiment, the invention further provides a method for ameliorating insulin-desensitization, glucose dysregulation, hyperglycemia or any combination thereof, using scaffolds comprising cells as described herein.

In another embodiment, the invention provides a glucose homeostasis rectifying cell population containing at least one GLUT4 overexpressing cell seeded and cultured on a scaffold as described herein. In some embodiments, transplanting the scaffold cell construct into a subject in need thereof, including but not limited to a subject afflicted with a diabetes, provides a glucose homeostasis rectifying effect.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

The term “subject” or “patient” refers to an animal which is the object of treatment, observation, or experiment. By way of example only, a subject includes, but is not limited to, a mammal, including, but not limited to, a human or a non-human mammal. Non-limiting examples of a non-human mammal include, primate, murine, bovine, equine, canine, ovine, or feline subject.

The term “biological sample” refers to a sample obtained from a subject, including, but not limited to, sample of biological tissue or fluid origin obtained in vivo or in vitro. Such samples can be, but are not limited to, body or bodily fluid (e.g., blood, blood plasma, serum, or urine), organs, tissues, fractions thereof, and cells isolated from mammals including, humans. Biological samples also may include sections of the biological sample including tissues (e.g., sectional portions of an organ or tissue). Biological samples may also include extracts from a biological sample, for example, an antigen from a biological fluid (e.g., blood or urine). In some embodiments, the biological sample is blood, tear, saliva, sweat, or urine. In some embodiments, the biological sample is obtained by noninvasive techniques, for example, from tears, saliva, sweat, or urine.

According to some embodiments, a method or a process of making a scaffold-cell construct configured to restore or maintain glucose levels is provided. In some embodiments, the method or process are directed to a scaffold-cell construct configured to restore or maintain glucose levels of: less than or equal to 100 mg/dL at fasting; and less than 140 mg/dL postprandial. In some embodiments, the method or process is directed to a scaffold-cell construct configured to restore or maintain glucose levels of: less than or equal to 120 mg/dL at fasting; and less than 160 mg/dL postprandial.

In some embodiments, the method or process comprise contacting a recombinant cell having increased GLUT4 activity with a scaffold comprising at least one biocompatible polymer. In some embodiments, a recombinant cell comprises an exogenous or endogenous GLUT4 polynucleotide, as mentioned hereinabove and such as exemplified herein.

In some embodiments, the method or process further comprise a differentiation step comprising seeding a recombinant cell on or in a scaffold for at least 3, at least 5, at least 7, or at least 9 days, and any value and range therebetween. In some embodiments, the method or process further comprise a differentiation step comprising seeding a recombinant cell on or in a scaffold for 3-9 days, 5-11 days, or 6-15 days. Each possibility represents a separate embodiment of the invention.

In some embodiments, the method or process, further comprise a validation step comprising detecting GLUT 4 expression or activity in at least a portion of the first cell population. In some embodiments, the method or process, further comprise a validation step comprising detecting glucose uptake activity in at least a portion of the first cell population. In some embodiments, the method or process, further comprise a validation step comprising detecting glucose homeostasis activity in at least a portion of the first cell population.

In some embodiments, the method or process, further comprise a validation step comprising determining the scaffold-cell construct to maintain glucose homeostasis or restoration to levels of: less than or equal to 100 mg/dL at fasting; and less than 140 mg/dL postprandial. In some embodiments, the method or process, further comprise a validation step comprising determining the scaffold-cell construct to maintain glucose homeostasis or restoration to levels of: less than or equal to 120 mg/dL at fasting; and less than 160 mg/dL postprandial.

As used herein, at least a portion comprises: at least 10%, at least 20%, at least 30%, at least 40%, at least 50%, at least 60%, at least 70%, at least 80%, at least 90%, or at least 95% of the first cell population, and any value and range therebetween. In some embodiments, a portion comprises 10-30%, 20-40%, 30-50%, 40-60%, 50-70%, 60-80%, 70-90%, or 80-100% if the first cell population. Each possibility represents a separate embodiment of the invention.

In some embodiments, the scaffold-cell construct comprises the first cell and second cell at a ratio of 10,000:1, 7,500:1, 5,000:1, 2,500:1, 1,500:1, 1,000:1, 500:1, 350:1, 200:1, 100:1, 50:1, 1:1, 1:50, 1:100, 1:200, 1:350, 1:500, 1:1,000, 1:2,500, 1:5,000, 1:7,500, or 1:10,000, and any value and range therebetween. In some embodiments, the scaffold-cell construct comprises the first cell and second cell at ratio ranging from 10,000:1 to 1:10,000, 7,500:1 to 1:7,500, 5,000:1 to 1:5,000, 2,500:1 to 1:2,500, 1,500:1 to 1:1,500, 1,000:1 to 1:1,000, 500:1 to 1:500, 350:1 to 1:350, 200:1 to 1:200, 100:1 to 1:100, or 50:1 to 1:50. Each possibility represents a separate embodiment of the invention.

### Treatment and Pharmaceutical Compositions

According to some embodiments, a pharmaceutical composition comprising scaffold-cell constructs and a pharmaceutically acceptable carrier, excipient, or adjuvants is provided. In some embodiments, the pharmaceutical composition comprises a therapeutically effective amount of the scaffold-cell constructs over-expressing GLUT4, for use in reducing glucose levels in a subject. In some embodiments, the composition is for use in reducing glucose levels to less than or equal to 100 mg/dL at fasting; and less than 140 mg/dL postprandial. In some embodiments, the pharmaceutical composition comprises a therapeutically effective amount of the scaffold-cell constructs over-expressing GLUT4, for use in reducing glucose levels in a subject. In some embodiments, the composition is for use in reducing glucose levels to less than or equal to 120 mg/dL at fasting; and less than 160 mg/dL postprandial.

In some embodiments, the pharmaceutical composition is for use in treating diabetes or other metabolic syndrome(s).

As used herein, the term a “metabolic syndrome, disease, disorder, or condition” refers to any disease or disorder characterized by excess abdominal fat, hypertension, abnormal fasting plasma glucose level or insulin resistance, high triglyceride levels, low high-density lipoprotein (HDL) cholesterol level, and any combination thereof. In some embodiments, the metabolic syndrome disorders which can be treated according to the present invention are diverse and will be easily understood by the skilled artisan. Without any limitation mentioned are obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance or insulin-resistance related. High risks of metabolic syndrome disease include, but are not limited to, obstructive sleep apnea, nonalcoholic steatohepatitis, chronic kidney disease, polycystic ovary syndrome and low plasma testosterone, erectile dysfunction, or both.

According to some embodiments, the use of the scaffold-cell construct of the present invention for preparation of a medicament for treating metabolic syndrome is provided.

As used herein, the terms “carrier”, “adjuvant” or “excipient” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents that may be useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The carrier may comprise, in total, from about 0.1% to about 99.99999% by weight of the pharmaceutical compositions presented herein.

As used herein, the terms “therapeutically active molecule” or “therapeutic agent” mean a molecule, group of molecules, complex or substance administered to an organism for diagnostic, therapeutic, preventative medical, or veterinary purposes. This term includes pharmaceuticals, e.g., small molecules, treatments, remedies, biologics, devices, and diagnostics, including preparations useful in clinical screening, prevention, prophylaxis, healing, imaging, therapy, surgery, monitoring, and the like. This term can also specifically include nucleic acids and compounds comprising nucleic acids that produce a bioactive effect, for example.

The term “therapeutically effective amount” refers to the concentration of cells within a scaffold-cell construct that over-express GLUT4 and are normalized to body weight (BW) that is effective to treat a disease or disorder in a mammal. The term “a therapeutically effective amount” refers to an amount effective, at dosages and for periods of time necessary, to achieve the desired therapeutic or prophylactic result. A physician or veterinarian of ordinary skill can readily determine and prescribe the effective amount of the bioactive agent required.

In some embodiments, a composition of the invention comprises pharmaceutically active agents. In some embodiments, pharmaceutically active agents are added prior to transplantation. Pharmaceutically active agents include but are not limited to any of the specific examples disclosed herein. Those of ordinary skill in the art will recognize also numerous other compounds that fall within this category and are useful according to the invention.

### Kit

According to some embodiments, provided herein is a kit comprising: a scaffold, and a first cell population of recombinant cells having increased GLUT4 activity. In some embodiments, the kit further comprises a cell culture medium. In some embodiments, the kit further comprises instruction for seeding the first cell population in or onto the scaffold. In some embodiments, the kit further comprises instruction for culturing the first cell population in or on the scaffold (e.g., using the cell culture medium). In some embodiments, kit further provides instructions for determining the % coverage of the first cell population in or on the scaffold. In some embodiments, the kit further comprises instructions for determining the GLUT4 activity of the first cell population. In some embodiment, the kit further comprises instructions for transplanting the scaffold-cell construct. In some embodiments, the kit further comprises a second cell population. In some embodiments, the kit further comprises instructions for seeding, and for culturing the second cell population. In some embodiments, the kit comprises a scaffold-cell construct comprising the first cell population. In some embodiments, the kit comprises the scaffold-cell construct comprising the first cell population and the second cell population. In some embodiments, the kit comprises the scaffold-cell construct comprising the first cell population, in a defined culture media devoid of animal-derived products or substances. In some embodiments, the kit comprises the scaffold-cell construct comprising the first cell population, and the second cell population, in a defined culture media devoid of animal-derived products or substances.

In another embodiment, the composition for reducing glucose levels is produced by simply mixing a carrier and the scaffold-cell construct wherein the scaffold comprises the first cell population attached thereto. In another embodiment, the composition for reducing glucose levels is produced by simply mixing a carrier and a scaffold-cell construct wherein the scaffold comprises the first cell population and the second cell population attached thereto.

In another embodiment, a kit for reducing glucose levels, comprises a first part that contains an effective amount of scaffolds, and a second part that contains an effective amount of a first cell population suspended in cell culture media. In another embodiment, the kit is for transplantation, and the first and second parts can be in solution form and are separately placed in independent packs (such as plastic bottles, tubes or glass bottles like ampoules).

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention, in some embodiments, include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cellular Transduction

L6 myoblasts were transduced using a pQCXIP retroviral plasmid, and then underwent positive selection with 10 μg/ml of Blasticidin HCl (Clbiochem), as previously described (Niu et al., (2010); Am. J. Physiol. Endocrinol. Metab.).

### Scaffold Fabrication

Porous biodegradable scaffolds were made using salt leaching technique to achieve pore sizes of 212-600 μm, out of poly-l-lactic acid (PLLA; Polysciences, Warrington) and polylactic glycolic acid (PLGA; Boehringer Ingelheim). A 5% (w/v) polymer solution of each polymer was prepared separately, by dissolving 0.5 gr polymer in 10 ml chloroform, in a small glass vial which was placed on a magnetic stirring plate overnight (ON). The polymers were then mixed together in a 1:1 ratio, to create a PLLA/PLGA solution. NaCl was sieved to 212-600 μm particles and 0.4 gr was poured into Teflon cylinder molds (18 mm internal diameter) and dissolved in 0.24 ml PLLA/PLGA solution. The Teflon molds were left ON to allow for chloroform evaporation, after which, the scaffolds were gently removed from the molds and placed into histology cassettes. The salt was leached by placing the histology cassettes in a beaker filled with distilled water, on a magnetic stirring plate; the water was exchanged every hour for 6-8 hours. The scaffolds were then removed from the histology cassettes, dried on a Kimwipe and frozen ON at −80° C., in a 50-ml tube. Finally, the scaffolds were lyophilized ON and kept dry under vacuum until use. One day prior to seeding, round pieces of 6 mm diameter were prepared using a biopsy punch (Miltex). These pieces were sterilized in 70% ethanol ON. Before seeding, the scaffolds were washed twice in phosphate buffer saline (PBS) (Sigma) and dried using vacuum.

### Cell Culture

L6WT and L6GLUT4 myoblasts were cultured in MEM Alpha medium (Biological Industries) supplemented with 10% of fetal bovine serum (FBS) (Hyclone), 1% Penstrep antibiotic (PS) (Biological Industries) and 0.1% Amphotericin B (Amp B) (anti-fungal) (Biological Industries). Cells are incubated in a 5% CO₂ humidified atmosphere at 37° C. L6GLUT4, were added directly in the culture plate with 2 μg/ml of Blasticidin HCl (Clbiochem), antibiotic for positive selection of GLUT4 over-expressing cells.

### Three-Dimensional Tissue Construction

Myoblasts were cultured up to 70% confluency and then washed with PBS and trypsionized with 2× Trypsin-EDTA (Biological Industries), centrifuged at room temperature (RT) at 0.2 rcf for 4 minutes and re-suspended in 6 μl of a 1:1 mixture of 15 mg/ml fibrinogen (Sigma) and a 10-20 U/ml thrombin (Sigma). The cell suspension, 0.25/0.5×10⁶ cells per scaffold, was seeded into PLLA/PLGA scaffolds and allowed to dry for 30 minutes at 37° C. and 5% CO₂, inside a 12-well plate. After solidification, 2 ml of growth medium (MEM Alpha, 10% FBS, 1% PS and 0.1% Amp B) were added to each well. After 2 days, the scaffolds were transferred to a new 12-well plate using a sterile forceps and 2 ml of differentiation medium (MEM Alpha, 2% FBS, 1% PS and 0.1% Amp B) were added to each well. For scaffolds seeded with L6GLUT4 cells, each well may optionally be supplemented with 2 μg/ml of Blasticidin HCl.

### Whole-Mount Scaffolds Staining

Scaffolds seeded with L6WT of L6GLU4 were cultured for 1 week, 2 weeks and 3 weeks in-vitro. Whole scaffolds were washed with PBS solution and then fixated in 4% paraformaldehyde for 20 min, the fixation solution was removed, and the scaffolds were washed with PBS. Next, the scaffolds were treated with 0.3% Triton X-100 for 10 min, in order to permeabilize the cells. Then the scaffolds were washed with PBS and soaked in blocking solution (10% FBS (v/v), 0.1% Triton (v/v), 1% Glycine (w/v)) ON at 4° C. The following day samples were incubated ON at 4° C., with a solution of primary antibodies diluted in blocking solution. Lastly, scaffolds were washed with PBS and incubated with secondary antibodies solution diluted in PBS for 3 hours at room temperature. Next, scaffolds were washed and stored in PBS until imaging.

### Glucose Uptake Evaluation by the Three-Dimensional Constructs

L6WT and L6GLUT4 cells were seeded on PLLA/PLGA scaffolds, at a seeding concentration of 0.25/0.5×10⁶ cells/scaffold, in a 12 well-plate, and cultured for up to 3 weeks in-vitro. After 3 weeks, the constructs were washed with PBS and then were serum-starved for 3 hours, followed by 30 minutes incubation in either absence or presence of 100 nM insulin (Basal and Insulin, respectively). 2-[3H]-deoxyglucose (2DOG; 0.1 mM; 2 Ci) was then added for additional 20 minutes. The uptake was then terminated by three rapid washes in ice-cold Krebs-Ringer-Phosphate (KRP) buffer. Following the last wash, the scaffolds were minced manually with fine scissors, vortexed and transferred to 500 μl of 1% SDS/1N NaOH solution at 85° C. for 1 hour, 50 μl samples were taken to measure cellular protein levels by BCA protein assay. The rest was added to 5 ml scintillation fluid in plastic minivials. 2DOG-associated radioactivity was counted using a beta-counter.

### BCA Assay

Protein levels for each construct were determined using BCA kit (Thermo Scientific). In a 96-well plate, the standards were prepared, in duplicates, out of a stock of 2 μg/μl of bovine serum albumin (BSA). Each construct sample that was 7.5 μl in duplicates, were added to 30 μl of 1% SDS/1N NaOH solution. Then, 150 μl of a mix of reagent A and reagent B (comprising 15 ml of reagent A and 300 μl of reagent B) were added to each well. The plate was incubated at 37° C. for 30 minutes and cooled at RT for 10 minutes. Afterwards the optical density (OD) was read at a wave length of 562 nm using a plate reader.

### Implant Procedure—Intra-Muscular Implantation in the Rectus Abdominis Muscle

The engineered construct was implanted after an in-vitro incubation of 2-3 weeks. Animals were anesthetized using a ketamine-xylazine cocktail (100 mg/Kg body weight (BW) and 10 mg/Kg BW, respectively) delivered with a 27-gauge needle via an i.p (intra-peritoneal) injection. A portion of the fur of the animal was removed by shaving and applying depilation cream, at the abdomen. A small incision in the skin was made allowing access to the linea-alba and surrounding tissue, where approximately 6 mm diameter defect was created by removing a full thickness tissue section from the abdominal wall. The constructs were sutured in place with four 8-0 silk sutures. Outer skin was closed and sutured using 5-0 AssuCryl surgical sutures. Animals were monitored closely for 1-2 hours to ensure full recovery.

### Glucose Tolerance Test—GTT Assay

Glucose basal level was established using a glucometer, by a blood test following 5-6 hour or overnight fasting of the animals. Then, the animals were injected i.p with a 10% (w/v) glucose solution. Blood samples were obtained at 15, 30, 45, 60, 75, 90 and 120 minutes following glucose administration.

### Example 1

### Morphology and Differentiation of Rat Skeletal Muscle Cells

Initially the myoblasts (skeletal muscle cells) were characterized for muscle morphology and ability to differentiate and form muscle fibers on the PLLA/PLGA scaffolds. L6WT and L6GLUT4 over-expressing cells were seeded at a concentration of 0.5×10⁶ cells/scaffold, and grown for 1, 2 and 3 weeks in-vitro. Following the incubation period, whole scaffolds were fixated and stained for different myogenic markers and the presence of the glucose transporter GLUT4; Desmin: a muscle-specific type III intermediate filament; Myosin heavy chain (MYH): a motor protein of muscle thick filaments; and Myogenin (MYOG): a muscle-specific transcription factor involved in the coordination of skeletal muscle development, differentiation and repair.

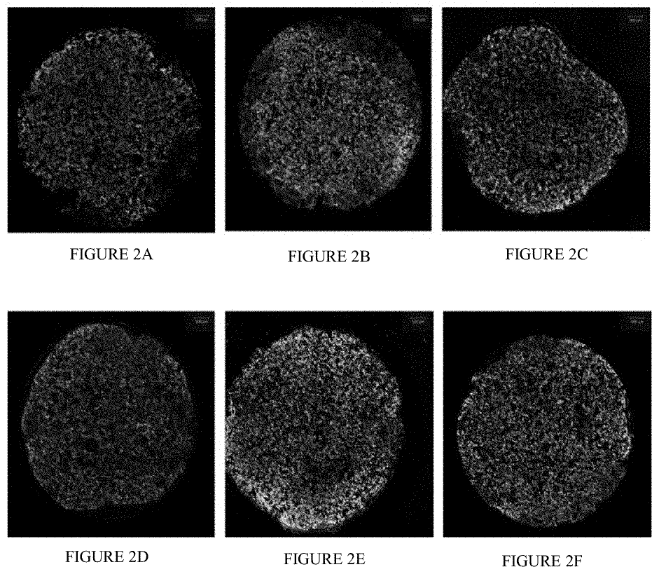

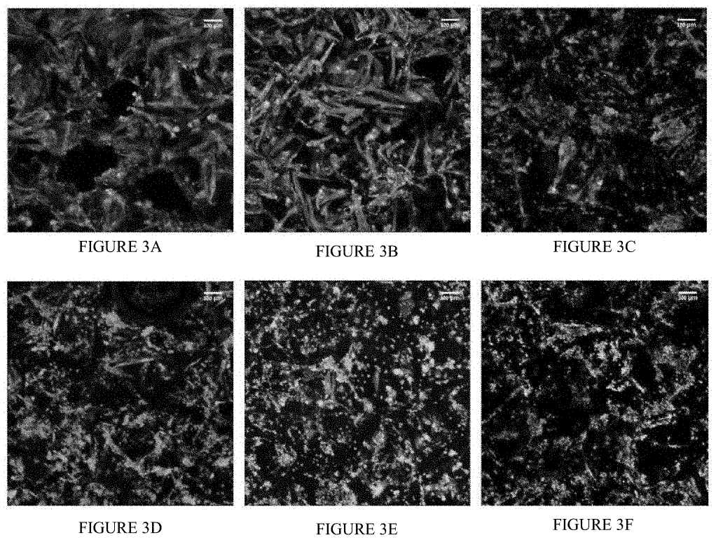

The inventors demonstrated that as early as 1-week post seeding, L6 cells expressed the different myogenic markers, meaning the cells undergone differentiation and formed fibers on PLLA/PLGA scaffolds (FIGS. 2 and 3). A greater GLUT4 expression signal was observed for the over-expressing cells (FIG. 2). After as early as 1 week of culturing, GLUT4 over-expressing cells were found to be positive for the muscular differentiation marker MYOG.

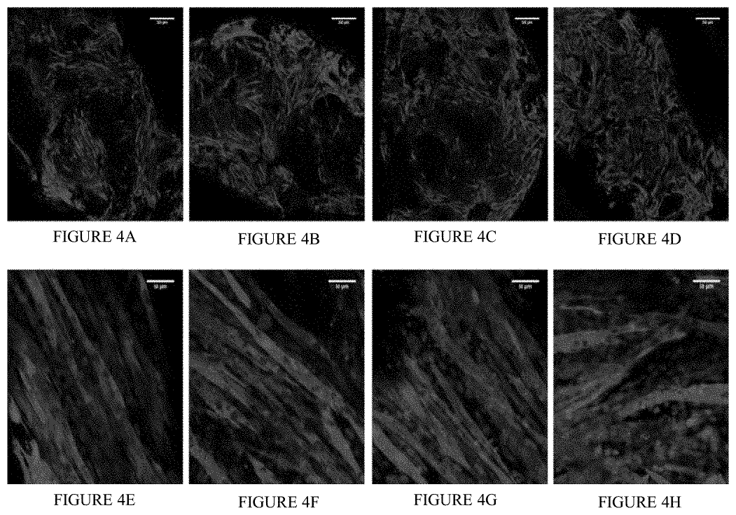

Cell differentiation was also analyzed after prolonged in-vitro culturing of 2 and 3 weeks. High expression of desmin, and the formation of defined and aligned muscle fibers throughout the whole scaffold, was observed for both WT and GLUT4 over-expressing cells (FIG. 4).

### Example 2

### Glucose Uptake Analysis In Vitro

To evaluate the glucose uptake ability of the three-dimensional constructs, a 2-[³H]-deoxyglucose (2DOG) uptake assay was performed. L6WT and L6GLUT4 myoblasts were seeded at a concentration of 0.25 or 0.5×10⁶ cells/well in 12 well-plate and cultured for 1, 2 or 3 weeks. After the in-vitro culturing periods, scaffolds were incubated in either the absence or presence of insulin (Basal and Insulin levels, respectively), and afterwards 2DOG was added to the samples. 2DOG is an analog of glucose and is radioactively labeled with tritium (3H). The 2DOG has the 2-hydroxyl group replaced with hydrogen, so it cannot undergo further glycolysis, and thus its uptake can be quantified. 2DOG associated radioactivity was counted using a beta-counter.

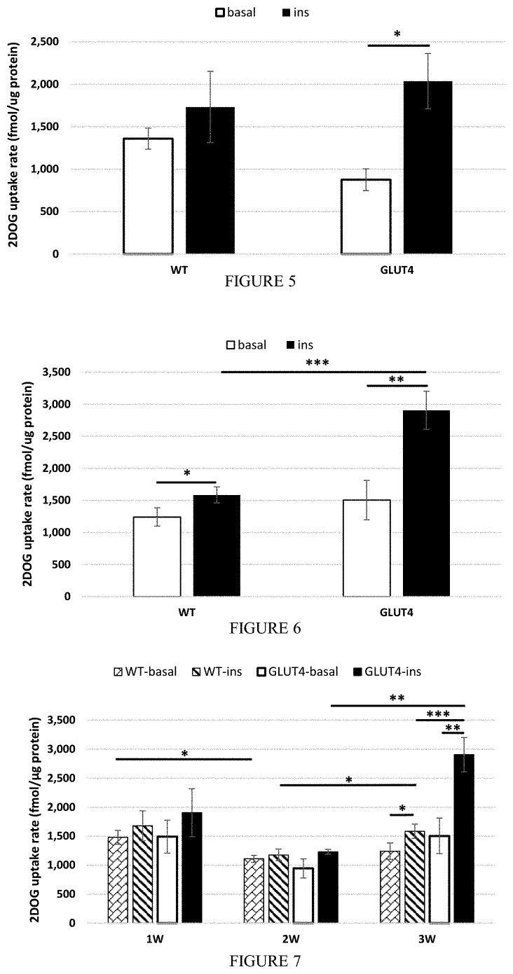

Upon seeding of 0.25×10⁶ cells per scaffold, the stimulated uptake by the L6GLUT4 cells was significantly higher compared to the basal uptake rate for L6GLUT4. No significant difference between the insulin stimulated uptake rates for the L6WT and L6GLUT4 constructs was observed (FIG. 5). However, upon increase of the cell concentration to 0.5×10⁶, a significantly higher insulin stimulated uptake rate was noted in the L6GLUT4 samples compared to L6WT (FIG. 6).

Next, the inventors wanted to determine whether higher cell concentration, will show higher glucose uptake for a shorter culturing period. Indeed, 3 weeks of culturing were shown to be preferable in terms of 2DOG uptake (FIG. 7). The inventors also noted that the uptake rate for 2 weeks was lower that for 1 week.

### Example 3

### Glucose Uptake In Vivo

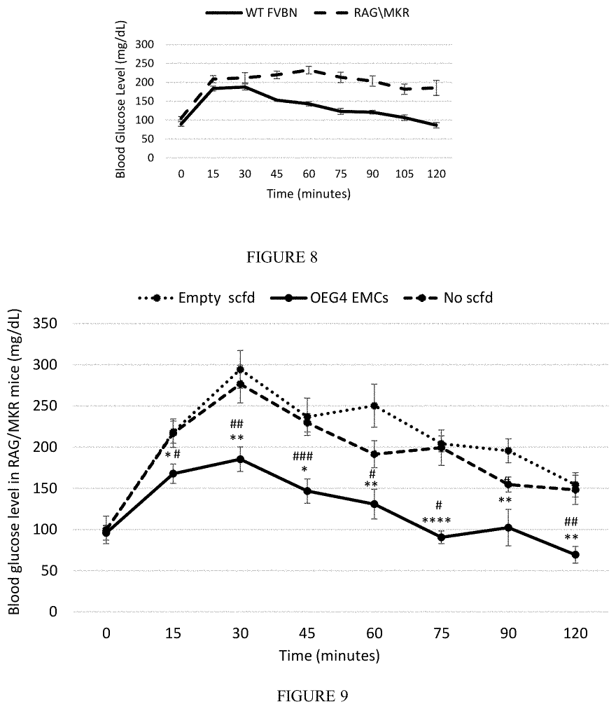

The animals used for the in-vivo experiments were RAG/MKR mice. These animals are immuno-deficient, having no mature B and T lymphocytes (RAG), and a genetically defective insulin receptor (MKR) leading to insulin-resistance and DM2 phenotype. Only males present the diabetic phenotype at the age of 8 weeks, and thus were implanted with a 6-mm scaffold, seeded with L6GLUT4 cells, in their abdominal muscle. In order to test the efficacy of the implants, a glucose tolerance test (GTT) was performed. The mice were injected i.p (intraperitoneal, injection into the body cavity) with a high dose (10% [w/v]) of glucose. It was shown by the inventors that due to their insulin resistance state, RAG/MKR mice, as oppose to their genetic background WT-FVBN counterparts, had a decreased tolerance for glucose and were unable to return to their basal glucose level (FIG. 8).

Eighteen (18) males at the age of 9-10 weeks were separated into 3 groups: 8 mice received constructs seeded with GLUT4 overexpressing L6 cells (OEG4 EMC), for control 6 mice did not receive any implant and 4 mice received empty scaffolds seeded with no cells and embedded with fibrin, to ensure that the implantation procedure does not affect the glycemic profile. The inventors demonstrated that mice which were implanted with GLUT4, showed better tolerance to glucose load compared to control mice, and their blood glucose levels returned to the basal-normal level faster compared to the control group (FIG. 9).

These data demonstrated that the in-vitro three-dimensional constructs of L6GLUT4 over-expressing cells, had higher glucose uptake ability compared to WT control. The inventors demonstrated using these in-vivo results, that overexpression of the GLUT4 transporter enhances overall increase in whole body glucose uptake and significantly improves blood glucose levels in DM2 a mammalian model organism.

### Example 4

### GLUT4-Overexpressing Satellite Cells

Stalitte cells (SC) are skeletal muscle precursor cells that are located beneath the basal lamina and comprise up to 1-5% of total skeletal muscle. They remain in quiescent state and can proliferate and undergo further differentiation following injury.

SCs were isolated from the tibialis muscle from the hind limbs of 8 week old C57BL6 mice. The hind limb was sterilized with 70% (v/v) ethanol and a cut was made at the ankle in order to expose the underline tissue. The outer skin and fascia were removed. The muscle was separated from the bone and cartilage and washed 3 times with cold DMEM supplemented with 1% PS. Next the muscle was dissected and minced, and the cut segments were placed under agitation for 1 hour at 37° C. in 0.25% trypsin-EDTA solution for enzymatic dissociation and then filtered through 100 μm membrane. The filtrate was centrifuged twice at room temperature (RT) for 10 min at 1,300 g. The pellet was re-suspended with BIO-AMF-2 medium and plated on a 0.1% gelatin coated plate and incubated overnight (O.N.) in a 5% CO₂ humidified atmosphere at 37° C. Forty eight (48) hours after harvest, myogenic cells were separated from fibroblasts using a plating technique based on their adherence to gelatin-coated surfaces. Cells were treated with 2× trypsin-EDTA, and centrifuged at RT for 10 minutes at 1,300 g. The pellet was re-suspended in DMEM supplemented with 10% FBS and 1% PS, and plated on a non-gelatin coated plate and incubated for 2 hours. This step was repeated twice. The fibroblasts adhered to the surface of the plate and the SC were suspended in the medium in the plate, which was collected from each incubation, centrifuged, re-suspended in BIOAMF-2 medium and plated on a gelatin coated plate.

### Morphological Characterization of Mouse Isolated SC

The inventors sought for suitable differentiation medium for the SC. For this SC were seeded at a concentration of 50,000 cells\well in a 24 well-plate. The cells were cultured in-vitro for a week, in 4 different mediums:
BIO-AMF-2, SC growth medium
DMEM medium, supplemented with 5% horse serum (HS) and 1% penstrep
DMEM medium, supplemented with 2% FBS and 1% penstrep
SKMCM medium, skeletal muscle cells growth medium, supplemented with 2% FBS and 1% penstrep

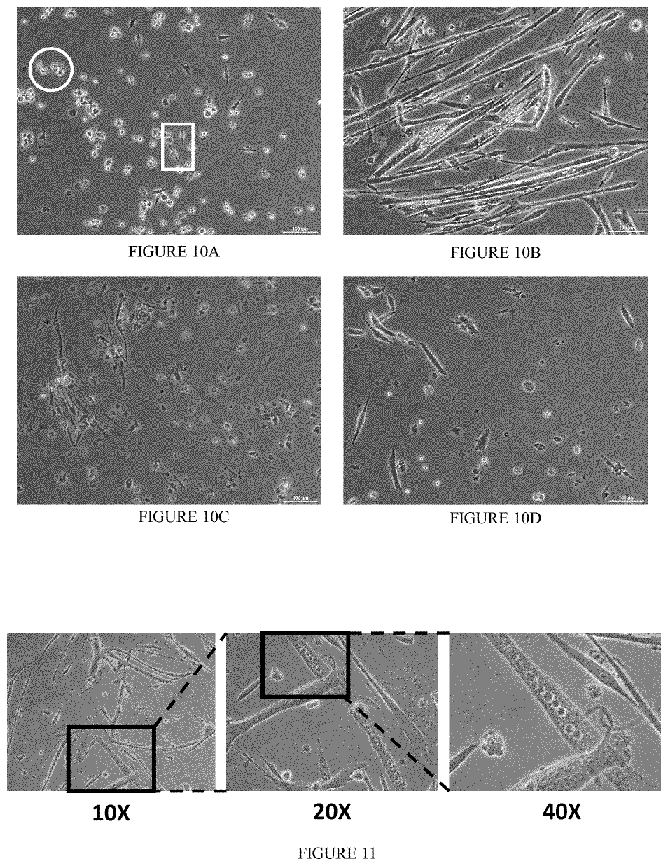

The SC has a single cell morphology (circle in FIG. 10A) and under high confluency or in the presence of differentiation factors it can differentiate into a myocyte (square in FIG. 10A). The inventors noted that the SC had the best differentiation rate with the DMEM supplemented with HS (FIG. 11B), as defined by the presence of multinucleated fibers. The differentiated C57-SC cells formed defined multinucleated elongated fibers (FIG. 11).

### Characterization of C57-SC Engineered Constructs

Isolated C57-SC were seeded at a concentration of 0.5×10⁶ and 1×10⁶ on PLLA\PLGA scaffolds and cultured in-vitro for 3 weeks in the chosen differentiation medium (DMEM supplemented with HS). After the culturing period the constructs were fixated and stained for desmin and MYOG, to assess the formation of muscle tissue.

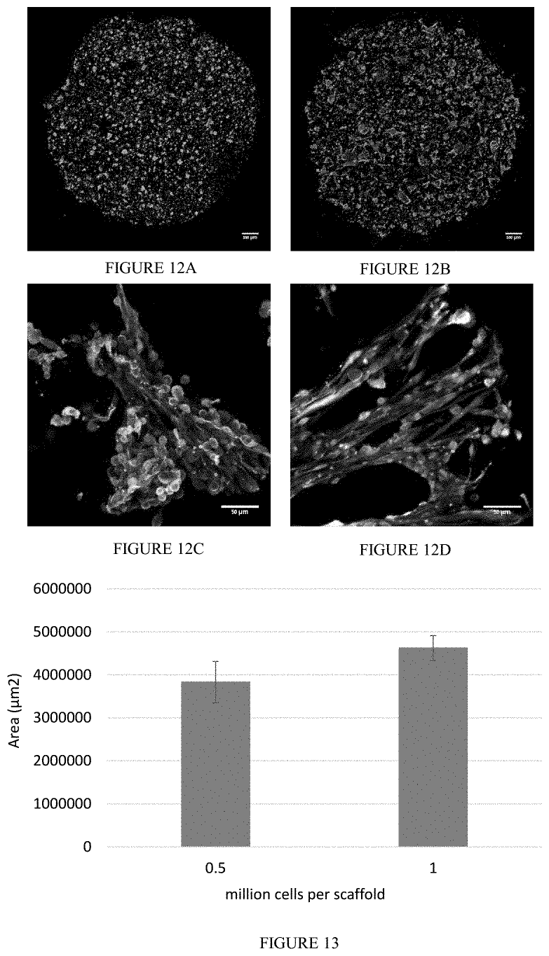

Differentiation and fiber formation were observed in both of the tested cell concentrations. The fibers for the higher initial concentration were more defined throughout the scaffold (FIGS. 12 and 14). However, quantification of the desmin signal showed no significant difference between the two concentrations (FIG. 13).

### GLUT4 Transduction

In order to establish stable over-expressing GLUT4 cells, C57-SCs were transduced with pQCXIB retroviral plasmid. The gene for GLUT4 was on the pQCXIB retroviral plasmid and the GP2-293 packing cells for viral production were used. The plasmid had the selection marker for Blasticidin antibiotic.

A day prior to the transfection, GP2-293 cells were seeded at a concentration of 4×10⁶ in a 10 cm plate. The following day 15 μg of the target plasmid, 15 μg of envelope plasmid and 9 μl of Xfext reagent were added to the packing cells to produce the viral particles. After 72 hours, retroviral supernatant was harvested and filtered through a 0.45 μm filter to remove cellular debris. The viral filtrate was diluted to the desired concentration and added to the cells, that were seeded a day before, for the transduction. The following day the viral particles were removed, and the cells were allowed to expand for 48 hours. Next, the inventors positively selected cells with 10 μg/ml of Blasticidin HCl antibiotic, according to the manufacturer's instructions (Clontech Laboratories, Inc., manual PT3132-1 (061113), Cat. No. 631530).

To further optimize transduction conditions which will provide higher yield of GLUT4 over-expressing cells, a large calibration assay was preformed, as follows.

The inventors tried 2 different envelope plasmids for the packing, 3 different virus concentrations, and 2 different facilitating agents, each at 4 concentrations. The conditions are detailed in table 1.

TABLE 1
Transduction calibration conditions

Envelope plasmid:

| | |
|---|---|
| p4070A (Ampho) | pgap70 (Eco) |

Virus concentration (% of total medium volume):

| | | |
|---|---|---|
| 25 | 33 | 50 |

Facilitating agent:

| Polybrene (μg/ml) (PB) | | | | Retronectin (μg/ml) (RN) | | | |
|---|---|---|---|---|---|---|---|
| 2 | 4 | 6 | 8 | 20 | 50 | 75 | 100 |

The resulting clones were analyzed to determine the over-expression, using immunofluorescent staining for GLUT4 transporter.

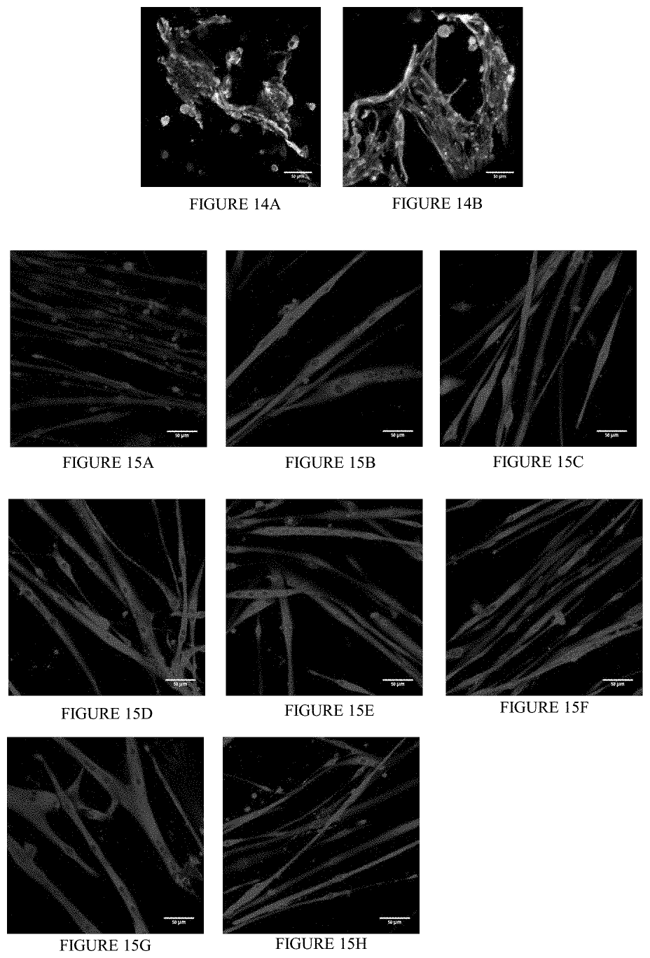

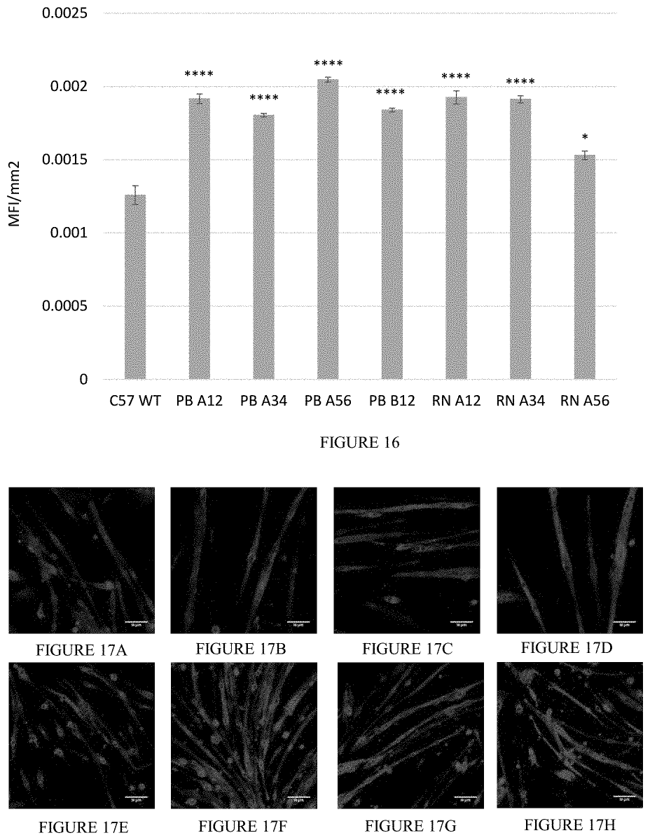

The description of the first batch of clones which was analyzed, is presented in table 2. FIG. 15 shows the staining of the GLUT4 transporter in these clones (table 2) compared to the WT cells (isolated and not transduced). FIG. 16 is a bar graph showing the quantification of the signal intensity of the immunofluorescent micrographs of FIG. 15.

TABLE 2
Description of the first batch of clones

| | |
|---|---|
| C57WT | PB A12 |
| | 2 μg/ml; 25% |
| RN A12 | PB A34 |
| 20 μg/ml; 25% | 2 μg/ml; 33% |
| RN A34 | PB A56 |
| 20 μg/ml; 33% | 2 μg/ml; 50% |
| RN A56 | PB B12 |
| 20 μg/ml; 50% | 4 μg/ml; 25% |

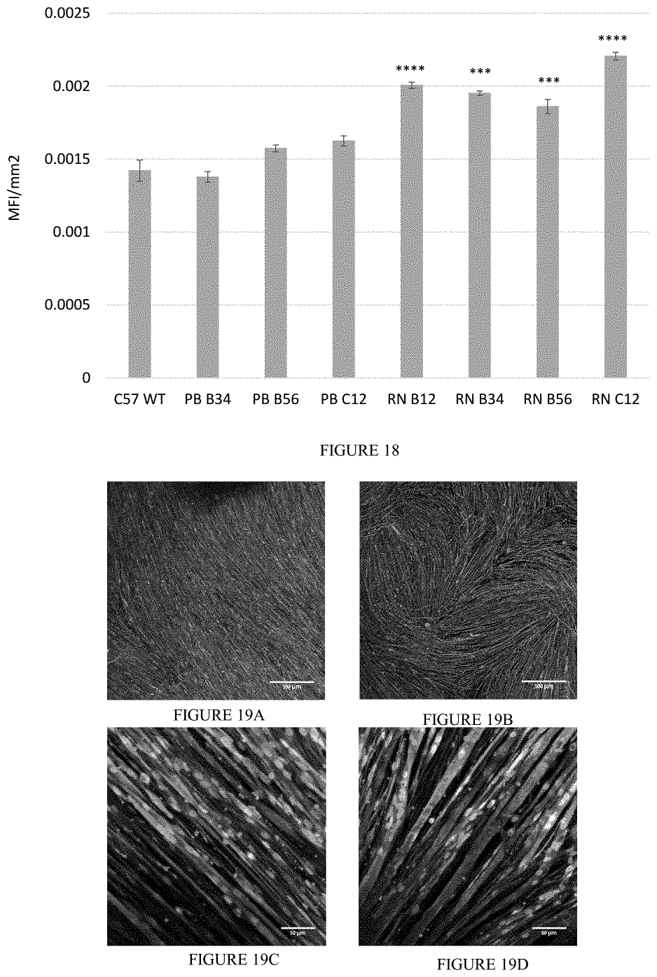

The description of the first batch of clones which was analyzed, is presented in table 3. FIG. 17 shows the staining of the GLUT4 transporter in these clones (table 3) compared to the WT cells (isolated and not transduced). FIG. 18 is a bar graph showing the quantification of the signal intensity of the immunofluorescent micrographs of FIG. 18.

TABLE 3
Description of the second batch of clones

| | |
|---|---|
| C57WT | PB B34 |
| | 4 μg/ml; 33% |
| RN B12 | PB B56 |
| 50 μg/ml; 25% | 4 μg/ml; 50% |
| RN B34 | PB C12 |
| 50 μg/ml; 33% | 6 μg/ml; 25% |
| RN B56 | RN C12 |
| 50 μg/ml; 50% | 75 μg/ml; 25% |

Fluorescent images and the quantification of the signals (FIGS. 15-18), showed that the inventors had successfully transduced naïve cells to over-express GLUT4.

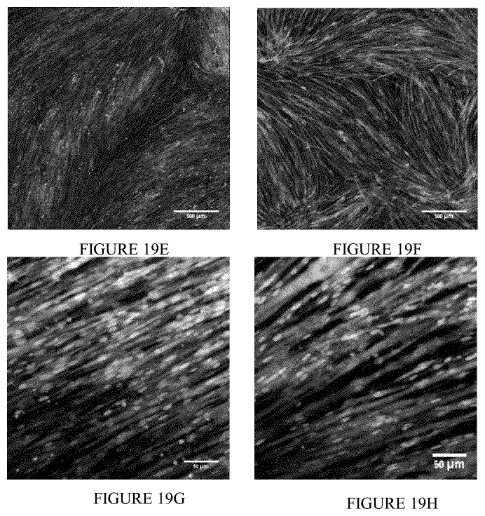

Clones PB-A12 and RN-B12 were then seeded in an optic 24 well-plate at a concentration of 50,000 cells\well and cultured for 1 week in-vitro. The formed myotubes were stained for the presence of the myogenic markers: desmin, MYH and MYOG. FIG. 19 shows defined myotubes expressing all of the tested markers.

The next step was to assess the ability of the transduced cells to uptake glucose. In order to calibrate the cell number required to preform a 2DOG uptake assay, as mentioned above, the inventors seeded: WT C57-SC, and the RN-A56 and PB-A34 clones, in a 12 well plate, in different concentrations (50,000, 100,00, 175,000 and 250,000 cells per well), cultured, and followed the cells for up to 10 days.

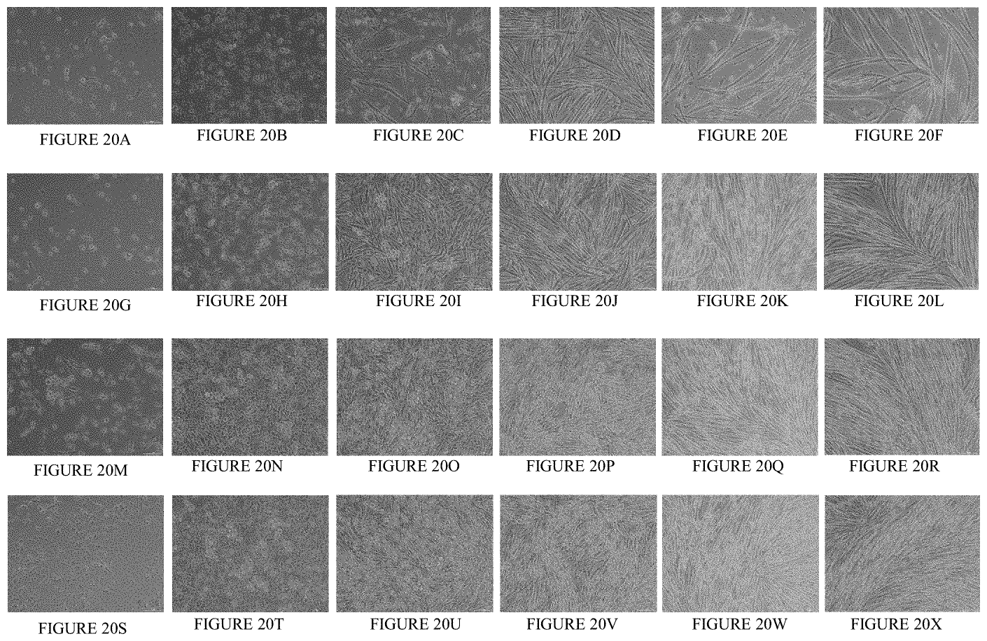

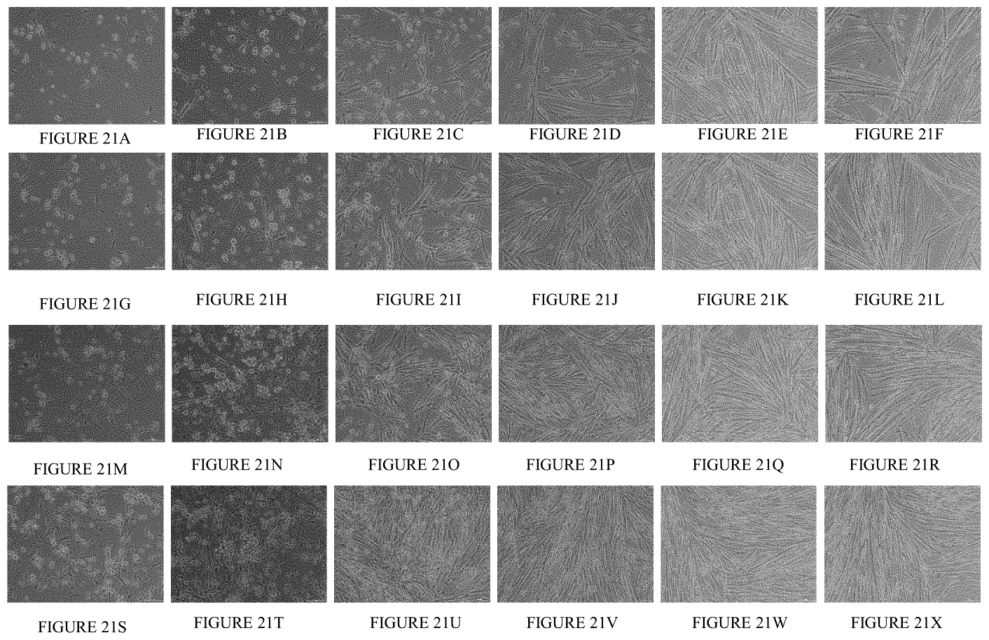

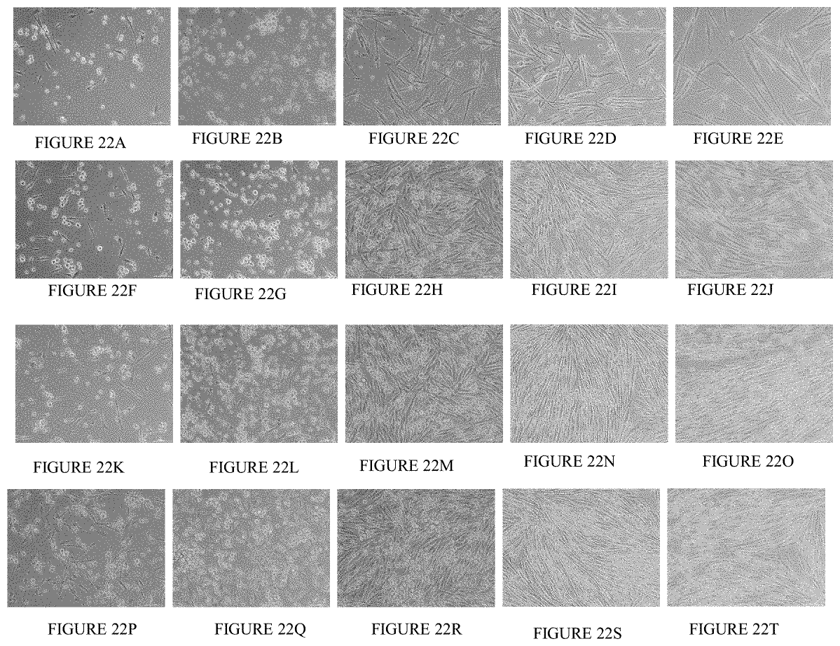

FIGS. 20-22 show that the transduced cells retained fusion ability and form defined myotubes. While full differentiation was observed for the 100,000 cells/well, for 2DOG uptake assay the inventors chose the 250,000 cells/well and 1 week of culturing, as these were the conditions for the L6 cells, mentioned hereinabove.

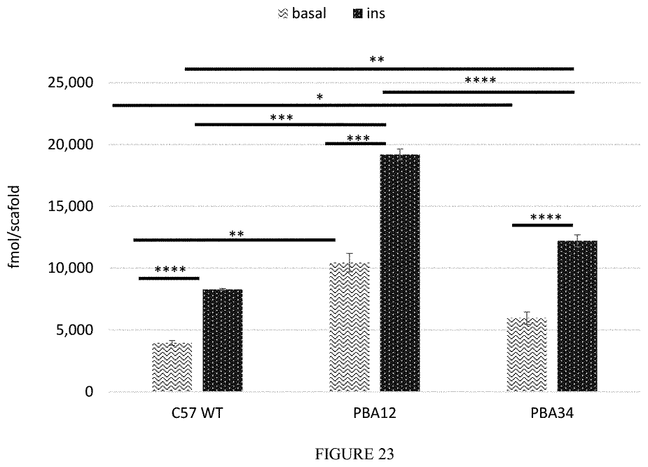

WT C57-SC and the clones PB-A12 and PBA34, were seeded at a concentration of 250,000 cells/well and cultured for 1 week, afterward a 2DOG uptake assay was performed. The uptake ability of 2 clones of the transduced cells compared to the WT cells is shown (FIG. 23). Both clones exhibited significantly higher basal and insulin stimulated glucose uptake. Clone PB-A12 demonstrated a higher glucose uptake rate than the PB-A34 rate.

The results show that it is feasible to isolate myogenic progenitor cells from a muscle biopsy, and further transduce these cells to overexpress GLUT4, which have relevant clinical usage for glycemic regulation.

### Example 5

### GLUT4-Overexpressing SCs Regulate Glycemic Homeostasis In Vivo

SCs isolated and transduced to overexpress GLUT4, as mentioned hereinabove e.g., as shown in FIGS. 16 and 18, are analyzed for glucose uptake ability. The best preforming clones, are then in vivo implanted (for example in DIO model C57BL6 mice). Glycemic values are recorded both under fasting conditions and postprandial, according to methods as described hereinabove (e.g., GTT). A SC clone which maintaines a glucose homeostasis at levels of less than or equal to 120 milligrams/Deciliter (mg/dL) at fasting; and less than 160 mg/dL postprandial, can potentially be used as a therapeutic agent for regulating glycemic homeostasis in vivo.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims which follow.

1. A scaffold-cell construct comprising:
a. a porous scaffold comprising at least one biocompatible polymer; and
b. a first cell population deposited on or in said scaffold comprising recombinant cells having increased glucose transporter type 4 (GLUT4) activity.

2. The scaffold-cell construct of claim 1, comprising recombinant cells having increased GLUT4 activity sufficient for maintaining glucose homeostasis at levels of:
i. less than or equal to 100 milligrams/Deciliter (mg/dL) at fasting; and
ii. less than 140 mg/dL postprandial.

3. The scaffold-cell construct of claim 1, comprising recombinant cells having increased GLUT4 activity sufficient for maintaining glucose homeostasis at levels of:
i. less than or equal to 120 mg/dL at fasting; and
ii. less than 160 mg/dL postprandial.

4. The scaffold-cell construct of claim 1, wherein said recombinant cells are induced to increase anyone of GLUT4 gene expression and/or membrane translocation by targeting the cellular GLUT4 translocation machinery pathway or insulin signal transduction.

5. The scaffold-cell construct of claim 4, wherein said insulin signal transduction is inhibition of GLUT4 degradation.

6. The scaffold-cell construct of claim 1, wherein said first cell population is selected from the group consisting of: skeletal myocyte-derived cell, cardiomyocyte-derived cell, adipocyte-derived cell, mesenchymal stem cell (MSC), embryonic stem cell (ESC), adult stem cell, differentiated ESC, differentiated adult Stem cell, or induced pluripotent Stem cell (iPSC).

7. The scaffold-cell construct of claim 1, wherein said first cell population is differentiated into myocyte, myoblast or myotube.

8. The scaffold-cell construct of claim 1, further comprising a second cell population for maintaining growth and survival of said first cell population.

9. The scaffold-cell construct of claim 1, comprising at least 1×10⁵ cells per mm of said polymer.

10. The scaffold-cell construct of claim 1, wherein said biocompatible polymer comprises any one of: (a) a polymer selected from a synthetic or natural material; and (b) one or more polymers selected from the group consisting of poly-1-lactic acid (PLLA), polylactic glycolic acid (PLGA), and any combination or derivative thereof.

11. (canceled)

12. The scaffold-cell construct of claim 1, wherein said biocompatible polymer comprises interconnected pores, wherein at least 80% of said pores have a diameter of between 200 and 600 microns.

13. The scaffold-cell construct of claim 12, wherein said PLLA and said PLGA are in a ratio of 3:1-1:3 w/w ratio.

14. (canceled)

15. (canceled)

16. (canceled)

17. (canceled)

18. A composition comprising the scaffold-cell construct of claim 1.

19. A method of making a scaffold-cell construct configured to restore or maintain glucose levels of:
i. less than or equal to 100 mg/dL at fasting; and
ii. less than 140 mg/dL postprandial;
the method comprising contacting recombinant cells having increased GLUT4 activity with a scaffold comprising at least one biocompatible polymer, and optionally further comprising a differentiation step comprising seeding said recombinant cells on or in said scaffold for at least 7 days, thereby fully differentiating said recombinant cells into recombinant skeletal muscle cells having increased GLUT4 activity.

20. (canceled)

21. The method of claim 19, further comprising a validation step comprising determining glucose homeostasis maintenance or restoration to levels of:
i. less than or equal to 100 mg/dL at fasting; and
ii. less than 140 mg/dL postprandial.

22. A method for reducing glucose levels in a subject in need thereof, the method comprising the steps of:
providing a scaffold-cell construct comprising a porous scaffold comprising at least one biocompatible polymer, and a first cell population deposited on or in said scaffold, said first cell population comprises recombinant cells having increased GLUT4 activity; and
grafting the subject with a therapeutically effective amount of said scaffold-cell construct, thereby reducing glucose levels in the subject.

23. The method of claim 22, wherein said reducing glucose levels comprises reducing glucose levels to:
i. less than or equal to 120 mg/dL at fasting; and
ii. less than 160 mg/dL postprandial.

24. The method of claim 22, wherein said reducing glucose levels comprises reducing glucose levels to:
i. less than or equal to 100 mg/dL at fasting; and
ii. less than 140 mg/dL postprandial.

25. The method of claim 22, wherein said grafting a subject is by autologous grafting or allogenous grafting.

26. The method of claim 22, wherein said subject is afflicted with diabetes mellitus or metabolic syndrome, and optionally wherein said metabolic syndrome is selected from: obesity, pre-diabetes and insulin resistance or related to insulin resistance.
